SEQUENCE LISTING (23 sequences)

SEQ ID NO: 1 - protein, 551 aa, Oryza sativa
  source 1..551: note = Oryza sativa Japonica Group
MLSPATTFSSSSSSSSPSRAHARAPTRFAVAASARAARFRPARAMAASDDPRGGRSVAVVGAGVSGLAAAYRLRKRGVQVTVFEAADRAGGKIRTNSEGGFIWDEGANTMTESELEASRLIDDLGLQGKQQYPNSQHKRYIVKDGAPTLIPSDPIALMKSTVLSTKSKLKLFLEPFLYEKSSRRTSGKVSDEHLSESVASFFERHFGKEVVDYLIDPFVAGTSGGDPESLSIRHAFPALWNLENKYGSVIAGAILSKLSTKGDSVKTGGASPGKGRNKRVSFSFHGGMQSLIDALHNEVGDGNVKLGTEVLSLACCCDGVSSSGGWSISVDSKDAKGKDLRKNQSFDAVIMTAPLSNVQRMKFTKGGVPFVLDFLPKVDYLPLSLMVTAFKKEDVKKPLEGFGALIPYKEQQKHGLKTLGTLFSSMMFPDRAPNDQYLYTSFIGGSHNRDLAGAPTAILKQLVTSDLRKLLGVEGQPTFVKHVHWRNAFPLYGQNYDLVLEAIAKMENNLPGFFYAGNNKDGLAVGNVIASGSKAADLVISYLESCTDQDN

SEQ ID NO: 2 - protein, 551 aa, synthetic construct
MLSPATTFSSSSSSSSPSRAHARAPTRFAVAASARAARFRPARAMAASDDPRGGRSVAVVGAGVSGLAAAYRLRKRGVQVTVFEAADRAGGKIRTNSEGGFIWDEGANTMTESELEASRLIDDLGLQGKQQYPNSQHKRYIVKDGAPTLIPSDPIALMKSTVLSTKSKLKLFLEPFLYEKSSRRTSGKVSDEHLSESVASFFERHFGKEVVDYLIDPFVAGTSGGDPESLSIRHAFPALWNLENKYGSVIAGAILSKLSTKGDSVKTGGASPGKGRNKRVSFSFHGGMQSLIDALHNEVGDGNVKLGTEVLSLACCCDGVSSSGGWSISVDSKDAKGKDLRKNQSFDAVIMTAPLSNVQRMKFTKGGVPFVLDFLPKVDYLPLSLMVTAFKKEDVKKPLEGFGALIPYKEQQKHGLKTLGTMFSSMMFPDRAPNDQYLYTSFIGGSHNRDLAGAPTAILKQLVTSDLRKLLGVEGQPTFVKHVHWRNAFPLYGQNYDLVLEAIAKMENNLPGFFYAGNNKDGLAVGNVIASGSKAADLVISYLESCTDQDN

SEQ ID NO: 3 - protein, 551 aa, synthetic construct
MLSPATTFSSSSSSSSPSRAHARAPTRFAVAASARAARFRPARAMAASDDPRGGRSVAVVGAGVSGLAAAYRLRKRGVQVTVFEAADRAGGKIRTNSEGGFIWDEGANTMTESELEASRLIDDLGLQGKQQYPNSQHKRYIVKDGAPTLIPSDPIALMKSTVLSTKSKLKLFLEPFLYEKSSRRTSGKVSDEHLSESVASFFERHFGKEVVDYLIDPFVAGTSGGDPESLSIRHAFPALWNLENKYGSVIAGAILSKLSTKGDSVKTGGASPGKGRNKRVSFSFHGGMQSLIDALHNEVGDGNVKLGTEVLSLACCCDGVSSSGGWSISVDSKDAKGKDLRKNQSFDAVIMTAPLSNVQRMKFTKGGVPFVLDFLPKVDYLPLSLMVTAFKKEDVKKPLEGFGALIPYKEQQKHGLKTLGTLFSSMMFPDRAPNDQYLYTSMIGGSHNRDLAGAPTAILKQLVTSDLRKLLGVEGQPTFVKHVHWRNAFPLYGQNYDLVLEAIAKMENNLPGFFYAGNNKDGLAVGNVIASGSKAADLVISYLESCTDQDN

SEQ ID NO: 4 - protein, 551 aa, synthetic construct
MLSPATTFSSSSSSSSPSRAHARAPTRFAVAASARAARFRPARAMAASDDPRGGRSVAVVGAGVSGLAAAYRLRKRGVQVTVFEAADRAGGKIRTNSEGGFIWDEGANTMTESELEASRLIDDLGLQGKQQYPNSQHKRYIVKDGAPTLIPSDPIALMKSTVLSTKSKLKLFLEPFLYEKSSRRTSGKVSDEHLSESVASFFERHFGKEVVDYLIDPFVAGTSGGDPESLSIRHAFPALWNLENKYGSVIAGAILSKLSTKGDSVKTGGASPGKGRNKRVSFSFHGGMQSLIDALHNEVGDGNVKLGTEVLSLACCCDGVSSSGGWSISVDSKDAKGKDLRKNQSFDAVIMTAPLSNVQRMKFTKGGVPFVLDFLPKVDYLPLSLMVTAFKKEDVKKPLEGFGALIPYKEQQKHGLKTLGTMFSSMMFPDRAPNDQYLYTSMIGGSHNRDLAGAPTAILKQLVTSDLRKLLGVEGQPTFVKHVHWRNAFPLYGQNYDLVLEAIAKMENNLPGFFYAGNNKDGLAVGNVIASGSKAADLVISYLESCTDQDN

SEQ ID NO: 5 - protein, 544 aa, Zea mays
MLALTASASSASSHPYRHASAHTRRPRLRAVLAMAGSDDPRAAPARSVAVVGAGVSGLAAAYRLRQSGVNVTVFEAADRAGGKIRTNSEGGFVWDEGANTMTEGEWEASRLIDDLGLQDKQQYPNSQHKRYIVKDGAPALIPSDPISLMKSSVLSTKSKIALFFEPFLYKKANTRNSGKVSEEHLSESVGSFCERHFGREVVDYFVDPFVAGTSAGDPESLSIRHAFPALWNLERKYGSVIVGAILSKLAAKGDPVKTRHDSSGKRRNRRVSFSFHGGMQSLINALHNEVGDDNVKLGTEVLSLACTFDGVPALGRWSISVDSKDSGDKDLASNQTFDAVIMTAPLSNVRRMKFTKGGAPVVLDFLPKMDYLPLSLMVTAFKKDDVKKPLEGFGVLIPYKEQQKHGLKTLGTLFSSMMFPDRAPDDQYLYTTFVGGSHNRDLAGAPTSILKQLVTSDLKKLLGVEGQPTFVKHVYWGNAFPLYGHDYSSVLEAIEKMEKNLPGFFYAGNNKDGLAVGSVIASGSKAADLAISYLESHTKHNNSH

SEQ ID NO: 6 - protein, 544 aa, synthetic construct
MLALTASASSASSHPYRHASAHTRRPRLRAVLAMAGSDDPRAAPARSVAVVGAGVSGLAAAYRLRQSGVNVTVFEAADRAGGKIRTNSEGGFVWDEGANTMTEGEWEASRLIDDLGLQDKQQYPNSQHKRYIVKDGAPALIPSDPISLMKSSVLSTKSKIALFFEPFLYKKANTRNSGKVSEEHLSESVGSFCERHFGREVVDYFVDPFVAGTSAGDPESLSIRHAFPALWNLERKYGSVIVGAILSKLAAKGDPVKTRHDSSGKRRNRRVSFSFHGGMQSLINALHNEVGDDNVKLGTEVLSLACTFDGVPALGRWSISVDSKDSGDKDLASNQTFDAVIMTAPLSNVRRMKFTKGGAPVVLDFLPKMDYLPLSLMVTAFKKDDVKKPLEGFGVLIPYKEQQKHGLKTLGTMFSSMMFPDRAPDDQYLYTTMVGGSHNRDLAGAPTSILKQLVTSDLKKLLGVEGQPTFVKHVYWGNAFPLYGHDYSSVLEAIEKMEKNLPGFFYAGNNKDGLAVGSVIASGSKAADLAISYLESHTKHNNSH

SEQ ID NO: 7 - protein, 502 aa, Glycine max
MASSATDDNPRSVKRVAVVGAGVSGLAAAYKLKSHGLDVTVFEAEGRAGGRLRSVSQDGLIWDEGANTMTESEIEVKGLIDALGLQEKQQFPISQHKRYIVKNGAPLLVPTNPAALLKSKLLSAQSKIHLIFEPFMWKRSDPSNVCDENSVESVGRFFERHFGKEVVDYLIDPFVGGTSAADPESLSMRHSFPELWNLEKRFGSIIAGALQSKLFAKREKTGENRTALRKNKHKRGSFSFQGGMQTLTDTLCKELGKDDLKLNEKVLTLAYGHDGSSSSQNWSITSASNQSTQDVDAVIMTAPLYNVKDIKITKRGTPFPLNFLPEVSYVPISVMITTFKKENVKRPLEGFGVLVPSKEQKNGLKTLGTLFSSMMFPDRAPSDLYLYTTFIGGTQNRELAQASTDELRKIVTSDLRKLLGAEGEPTFVNHFYWSKGFPLYGRNYGSVLQAIDKIEKDLPGFFFAGNYKGGLSVGKAIASGCKAADLVISYLNSASDNTVPDK

SEQ ID NO: 8 - protein, 502 aa, synthetic construct
MASSATDDNPRSVKRVAVVGAGVSGLAAAYKLKSHGLDVTVFEAEGRAGGRLRSVSQDGLIWDEGANTMTESEIEVKGLIDALGLQEKQQFPISQHKRYIVKNGAPLLVPTNPAALLKSKLLSAQSKIHLIFEPFMWKRSDPSNVCDENSVESVGRFFERHFGKEVVDYLIDPFVGGTSAADPESLSMRHSFPELWNLEKRFGSIIAGALQSKLFAKREKTGENRTALRKNKHKRGSFSFQGGMQTLTDTLCKELGKDDLKLNEKVLTLAYGHDGSSSSQNWSITSASNQSTQDVDAVIMTAPLYNVKDIKITKRGTPFPLNFLPEVSYVPISVMITTFKKENVKRPLEGFGVLVPSKEQKNGLKTLGTMFSSMMFPDRAPSDLYLYTTMIGGTQNRELAQASTDELRKIVTSDLRKLLGAEGEPTFVNHFYWSKGFPLYGRNYGSVLQAIDKIEKDLPGFFFAGNYKGGLSVGKAIASGCKAADLVISYLNSASDNTVPDK

SEQ ID NO: 9 - DNA, 1403 bp, synthetic construct
  source 1..1403: mol_type = other DNA
ctcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcgctctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaag

SEQ ID NO: 10 - DNA, 243 bp, synthetic construct
  source 1..243: mol_type = other DNA
atggccactgcaacctccgcatcactgttttctaccgtgtctagcagctattcaaaggcttcttcaattccacactcccggctccaatcggttaaatttaattccgtcccgtcgtttacagggctgaagtctacttcgttgatttctggctcggattcctcgtcgcttgctaagacgttgcgggggtcggtgaccaaagcccaaacttcagacaaaaagccatacgggtttaagataaacgcg

SEQ ID NO: 11 - DNA, 1659 bp, synthetic construct
  source 1..1659: mol_type = other DNA
atgttgtctcccgcaacgactttttcctcatcatcatcttcgagctcgccgtccagagcccacgctagagccccaacaagatttgccgttgctgcatcagccagagcagcgaggttccggcctgctagagctatggctgcgtctgacgacccgaggggtgggagaagcgttgcggtcgtgggggccggggtgtcgggactggcggccgcttacagattgcgcaagcggggtgtccaggttacggtcttcgaggctgctgacagggcgggtggaaagataaggaccaatagcgaaggtggcttcatctgggatgaaggcgccaacacgatgactgagtccgaacttgaagcaagccggctgattgatgatcttggcctccagggaaagcagcagtaccctaacagccagcacaaacgctacattgttaaagacggagcgcctacgctcatcccttccgatccaattgctctcatgaaatctaccgtgctctcaaccaaatcaaagctgaagttgttcttggagccctttttgtatgaaaagagctcacgcagaacttccgggaaggtgtcagatgagcatttgtccgagtcagtggcctctttttttgagaggcacttcgggaaggaagtggttgattaccttatcgacccttttgtggcaggcacgtcaggtggggatcccgaaagcctgtcgattcggcacgcttttcccgcgctttggaatttggaaaacaaatacggatccgttattgctggcgccatcctcagcaaactttctacgaaaggggactctgtgaagacaggcggagcttcgccaggcaaaggccgcaataagcgggtgtcgttttcctttcatggagggatgcaaagccttatcgatgccctccacaatgaggtgggtgacggtaacgtgaaactcggcaccgaggttctttctttggcgtgttgctgcgatggagtgtcgtctagcggtggatggtctatatctgtcgattcaaaggacgctaaggggaaagatctccggaagaaccaatcattcgacgcggtcatcatgacagccccgctttcgaacgtccagaggatgaagtttactaagggaggggttcctttcgttcttgattttcttcccaaggtcgattatcttccgcttagcctcatggtcaccgcgtttaaaaaagaggatgtcaaaaagccccttgaaggttttggtgcacttattccgtacaaagagcaacaaaaacacggtcttaaaacgctggggaccatgttctcctcaatgatgttcccggatagggctcctaatgaccagtatctttatacatcaatgattggcggctcgcataacagggatctggcaggtgcccccacggccattctcaaacagttggtgacttccgacctgagaaaacttctgggagttgagggacagcccaccttcgtcaaacacgtgcactggaggaacgctttccctttgtacgggcaaaattatgatttggtgttggaagcaatcgctaaaatggagaacaaccttcccggcttcttttatgcgggtaataataaagatgggcttgcagttggcaatgttattgcatccggttcgaaggctgccgatcttgttatttcgtatctggaaagctgtacagatcaggacaactgatag

SEQ ID NO: 12 - DNA, 256 bp, synthetic construct
  source 1..256: mol_type = other DNA
cccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 13 - DNA, 613 bp, synthetic construct
  source 1..613: mol_type = other DNA
ggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagga

SEQ ID NO: 14 - DNA, 555 bp, synthetic construct
  source 1..555: mol_type = other DNA
atgtcgcccgagcgcaggcccgtcgagatccgcccggccacagcagccgacatggctgcggtgtgcgacatcgtcaaccactacatcgaaacgtccaccgtgaacttccgcaccgagccgcagacgccgcaggagtggatcgacgaccttgagcgcctccaggaccgctacccctggctcgtcgcggaggtcgaaggcgtcgtggccggcatcgcctacgccgggccgtggaaggctcgcaacgcctacgactggaccgtcgagagcaccgtctacgtgtcccaccgccaccagcgcctgggcctcggctccacgctgtacacgcacctgctcaagtcgatggaggcgcaaggcttcaagagcgtggtcgccgtcatcggcctcccgaacgatccctccgtgaggctccacgaagccctgggctacaccgctcggggcacgctgcgcgcagccggctacaagcacggtggctggcacgacgtcgggttctggcagagggacttcgagctgcccgcgcctccgcgcccggttcgccccgtgacccagatttgatag

SEQ ID NO: 15 - DNA, 174 bp, synthetic construct
  source 1..174: mol_type = other DNA
tttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagat

SEQ ID NO: 16 - DNA, 517 bp, synthetic construct
  source 1..517: mol_type = other DNA
ccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgta

SEQ ID NO: 17 - DNA, 555 bp, synthetic construct
  source 1..555: mol_type = other DNA
atgagcccagaaagacgtccagtggagatccgtcctgcaaccgccgccgatatggcagccgtgtgtgatatagttaaccattacatcgaaaccagcacagttaatttcaggaccgaacctcaaactcctcaagaatggatagatgaccttgaaaggctccaggatcgttatccctggttggtggccgaagtagagggcgtcgtcgccggaatcgcatatgctggcccttggaaggcaaggaacgcttatgattggactgtggagagcacagtgtacgtgagccacaggcaccaaaggctcggattgggttccacactctatacacatctgctcaagtcaatggaagctcaggggtttaaatcagtagtcgcagtcataggtttgcctaatgatcccagtgtaagactccacgaggcactcggatacacagctcgcggaactcttagggccgccggatataaacatggtggctggcacgacgtgggcttttggcaacgcgactttgaactccctgcccctccaaggccagtgaggccagtcacacaaatttgataa

SEQ ID NO: 18 - DNA, 641 bp, synthetic construct
  source 1..641: mol_type = other DNA
agctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatca

SEQ ID NO: 19 - DNA, 1646 bp, synthetic construct
  source 1..1646: mol_type = other DNA
tgatgattcttgttgttgtagttctttttaaaagtcccacctgagcctctatagactctgattctcttttaagttactattttcaccgctctctaataaggctcgtgattttttgggagccatactgtatactcgccggccctctcacgatgttgttcaattcacagactaaatttgatttatctatttcgccaaaacataacttcattaaaaaatgttctccaaataactaaacgaattaaataaaagaaacctttcatgtaaagttaaaggtatgagactttaagggcagttgctgaacattcgtaacacatgggagaacaatagagaaagttgaaaagaaacgtagcatatagaaaaattatctttgtaaccaagttgatttagaaaaatatcactatttgtgaaaaatactagatcagtttattattacttttttttttttgtatattcacaaatatcatattcatatagaagaaaataaacaaagttgtaaaaatctggcatttaaaataaaattgaacacttcaatttatttcctttcataataataattttggcataagatatttgcaaattgatctggttcggtatggtcgacaaaataattttccacgctacccttccagccgtccattcactatttgccctcaacgttaccaaataacggtccagattcctagggcaagatctaacggttagcaagtaaagtcgtaccatcagaaagaataacaattctttcacaaagtaaacataaccaacggttaacaagttcttagggttaaatcagtaagatccaacggatattaaattgcaaggcccaaatagtttttttgcagcagataataactcgtccccactggcgagtgacgaccgagactctgtgaccctatttttcgagacgataaaagggcaaacaatcgctcttttcaaagctcgcctcttcaccacagagaaaacttcgtctctcttctctgcttcgccctctcatttcctgtgagataaaggcggagtctctctccagttattttgctcatccatcgattcttaggtatgactcgtttctctcagatctgtgattctttataatctcgtcgttcttcaaatcattgttatattcgtttcttcgatctgtgttttttagatctgtaaggtaaatgagacgtttcgatctgtagatctgattgttatattgatagattatgttatctgctttgcttaaagtccgatcggaatgttttgtgctcattgtcgaatatctgatgtatcggtttcatagatctgcttctttttgtgcgtttcgttgatctgataatcttctagtgatcaaaatcgtttggatctgttgactttagtttaaaatgtatccgatctgatgtcgaggcttcattattggaagttgttattgttgtaatcctgatttaagttgctgttcttaaatttatatgatctttgcgttataatatgacatggtagatcttggttcatggttcactgttttccaataaacttggtttgtttggttggatagcgttctgtgatacgaccatgtcttgtgttggataagaattctctgaatttccttggctggtttgtagtatgttattcacgtctggtttctcatcaatgattatgtgattttgcag

SEQ ID NO: 20 - DNA, 1659 bp, synthetic construct
  source 1..1659: mol_type = other DNA
atgctgtccccagcaaccaccttcagcagttcttcttcatctagctcaccctctagggcccatgcacgcgcccctactcgtttcgctgtagccgcctccgctagagcagcacgcttcagacctgcaagagccatggccgcctcagatgacccaaggggagggaggtccgttgcagtggttggagccggcgtaagcggcctcgctgccgcataccgcttgagaaaaagaggtgtacaggtgacagtgttcgaggcagctgaccgtgccggcggtaaaatccgtaccaatagcgaaggaggctttatatgggacgagggtgctaacactatgaccgaaagtgaactggaggcaagcaggctcatagatgatttgggactgcaaggcaagcaacaatatcccaattctcagcataaacgctatattgtaaaggatggcgctcctacactgattcccagcgacccaatagctttgatgaaaagcactgtgctcagcactaaatccaaactgaagctcttccttgaaccatttctctatgagaagagtagccgcagaacatcaggtaaggtgagcgatgaacacctgagcgagtcagtggctagcttcttcgagcgtcactttggcaaggaggtagtggattatctcattgatccatttgtggccggtacctcagggggagatcccgaatccctcagtattcgccacgcatttcctgctctctggaaccttgaaaataagtacggatctgtcatagctggggctattctcagtaagctctccactaagggtgattcagttaagacaggtggtgctagcccagggaaaggtagaaataaacgtgtgtcattcagcttccatgggggaatgcaaagtctgatagatgcattgcataatgaagtgggtgatgggaacgtgaaactcggaacagaggtgctttcacttgcttgctgctgcgatggggtttcttccagcggcggttggtctatctccgtagattcaaaggatgctaaggggaaggacctgcgtaaaaatcagtccttcgacgctgtcataatgaccgcacctttgagtaacgttcaaaggatgaaattcaccaaggggggagtaccctttgtccttgatttcttgcctaaggttgattacctccccctgagtcttatggtaactgcctttaaaaaagaagatgtgaaaaaaccactcgagggtttcggtgctcttataccttataaagaacaacagaaacatgggctgaaaactctcggcaccatgttctccagcatgatgttcccagatagggctccaaacgatcagtatctctacacatctatgatcgggggaagtcataatcgtgaccttgccggggcccccacagccatattgaagcaattggtaactagcgatctcagaaaacttttgggagtcgaaggccaaccaacttttgttaaacatgtgcactggcgtaatgcatttcccctgtatggtcaaaattacgaccttgtactggaagccatcgcaaagatggaaaacaatttgcctggcttcttttacgccgggaacaacaaggacggacttgctgttgggaacgtgatagccagcggttcaaaggccgccgacctggtaatctcctacctggagagttgtaccgatcaggataactagtga

SEQ ID NO: 21 - DNA, 253 bp, synthetic construct
  source 1..253: mol_type = other DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 22 - DNA, 11421 bp, synthetic construct
  source 1..11421: mol_type = other DNA
gtttacccgccaatatatcctgtcaaacactgatagtttaaactgaaggcgggaaacgacaatctgatccaagctcaagctgctaagcttatcctcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcgctctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagcctaggatggccactgcaacctccgcatcactgttttctaccgtgtctagcagctattcaaaggcttcttcaattccacactcccggctccaatcggttaaatttaattccgtcccgtcgtttacagggctgaagtctacttcgttgatttctggctcggattcctcgtcgcttgctaagacgttgcgggggtcggtgaccaaagcccaaacttcagacaaaaagccatacgggtttaagataaacgcgatgttgtctcccgcaacgactttttcctcatcatcatcttcgagctcgccgtccagagcccacgctagagccccaacaagatttgccgttgctgcatcagccagagcagcgaggttccggcctgctagagctatggctgcgtctgacgacccgaggggtgggagaagcgttgcggtcgtgggggccggggtgtcgggactggcggccgcttacagattgcgcaagcggggtgtccaggttacggtcttcgaggctgctgacagggcgggtggaaagataaggaccaatagcgaaggtggcttcatctgggatgaaggcgccaacacgatgactgagtccgaacttgaagcaagccggctgattgatgatcttggcctccagggaaagcagcagtaccctaacagccagcacaaacgctacattgttaaagacggagcgcctacgctcatcccttccgatccaattgctctcatgaaatctaccgtgctctcaaccaaatcaaagctgaagttgttcttggagccctttttgtatgaaaagagctcacgcagaacttccgggaaggtgtcagatgagcatttgtccgagtcagtggcctctttttttgagaggcacttcgggaaggaagtggttgattaccttatcgacccttttgtggcaggcacgtcaggtggggatcccgaaagcctgtcgattcggcacgcttttcccgcgctttggaatttggaaaacaaatacggatccgttattgctggcgccatcctcagcaaactttctacgaaaggggactctgtgaagacaggcggagcttcgccaggcaaaggccgcaataagcgggtgtcgttttcctttcatggagggatgcaaagccttatcgatgccctccacaatgaggtgggtgacggtaacgtgaaactcggcaccgaggttctttctttggcgtgttgctgcgatggagtgtcgtctagcggtggatggtctatatctgtcgattcaaaggacgctaaggggaaagatctccggaagaaccaatcattcgacgcggtcatcatgacagccccgctttcgaacgtccagaggatgaagtttactaagggaggggttcctttcgttcttgattttcttcccaaggtcgattatcttccgcttagcctcatggtcaccgcgtttaaaaaagaggatgtcaaaaagccccttgaaggttttggtgcacttattccgtacaaagagcaacaaaaacacggtcttaaaacgctggggaccatgttctcctcaatgatgttcccggatagggctcctaatgaccagtatctttatacatcaatgattggcggctcgcataacagggatctggcaggtgcccccacggccattctcaaacagttggtgacttccgacctgagaaaacttctgggagttgagggacagcccaccttcgtcaaacacgtgcactggaggaacgctttccctttgtacgggcaaaattatgatttggtgttggaagcaatcgctaaaatggagaacaaccttcccggcttcttttatgcgggtaataataaagatgggcttgcagttggcaatgttattgcatccggttcgaaggctgccgatcttgttatttcgtatctggaaagctgtacagatcaggacaactgatagtctagacccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcatgcatcaatacttttgcgtattggctagagcagcttgccggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggaaccatgtcgcccgagcgcaggcccgtcgagatccgcccggccacagcagccgacatggctgcggtgtgcgacatcgtcaaccactacatcgaaacgtccaccgtgaacttccgcaccgagccgcagacgccgcaggagtggatcgacgaccttgagcgcctccaggaccgctacccctggctcgtcgcggaggtcgaaggcgtcgtggccggcatcgcctacgccgggccgtggaaggctcgcaacgcctacgactggaccgtcgagagcaccgtctacgtgtcccaccgccaccagcgcctgggcctcggctccacgctgtacacgcacctgctcaagtcgatggaggcgcaaggcttcaagagcgtggtcgccgtcatcggcctcccgaacgatccctccgtgaggctccacgaagccctgggctacaccgctcggggcacgctgcgcgcagccggctacaagcacggtggctggcacgacgtcgggttctggcagagggacttcgagctgcccgcgcctccgcgcccggttcgccccgtgacccagatttgatagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcactagtgaattcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttag

SEQ ID NO: 23 - DNA, 11744 bp, synthetic construct
  source 1..11744: mol_type = other DNA
gtttacccgccaatatatcctgtcaaacactgatagtttaaactgaaggcgggaaacgacaatctgatccaagctcaagctgctaagcttccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaatgagcccagaaagacgtccagtggagatccgtcctgcaaccgccgccgatatggcagccgtgtgtgatatagttaaccattacatcgaaaccagcacagttaatttcaggaccgaacctcaaactcctcaagaatggatagatgaccttgaaaggctccaggatcgttatccctggttggtggccgaagtagagggcgtcgtcgccggaatcgcatatgctggcccttggaaggcaaggaacgcttatgattggactgtggagagcacagtgtacgtgagccacaggcaccaaaggctcggattgggttccacactctatacacatctgctcaagtcaatggaagctcaggggtttaaatcagtagtcgcagtcataggtttgcctaatgatcccagtgtaagactccacgaggcactcggatacacagctcgcggaactcttagggccgccggatataaacatggtggctggcacgacgtgggcttttggcaacgcgactttgaactccctgcccctccaaggccagtgaggccagtcacacaaatttgataaagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaattgatgattcttgttgttgtagttctttttaaaagtcccacctgagcctctatagactctgattctcttttaagttactattttcaccgctctctaataaggctcgtgattttttgggagccatactgtatactcgccggccctctcacgatgttgttcaattcacagactaaatttgatttatctatttcgccaaaacataacttcattaaaaaatgttctccaaataactaaacgaattaaataaaagaaacctttcatgtaaagttaaaggtatgagactttaagggcagttgctgaacattcgtaacacatgggagaacaatagagaaagttgaaaagaaacgtagcatatagaaaaattatctttgtaaccaagttgatttagaaaaatatcactatttgtgaaaaatactagatcagtttattattacttttttttttttgtatattcacaaatatcatattcatatagaagaaaataaacaaagttgtaaaaatctggcatttaaaataaaattgaacacttcaatttatttcctttcataataataattttggcataagatatttgcaaattgatctggttcggtatggtcgacaaaataattttccacgctacccttccagccgtccattcactatttgccctcaacgttaccaaataacggtccagattcctagggcaagatctaacggttagcaagtaaagtcgtaccatcagaaagaataacaattctttcacaaagtaaacataaccaacggttaacaagttcttagggttaaatcagtaagatccaacggatattaaattgcaaggcccaaatagtttttttgcagcagataataactcgtccccactggcgagtgacgaccgagactctgtgaccctatttttcgagacgataaaagggcaaacaatcgctcttttcaaagctcgcctcttcaccacagagaaaacttcgtctctcttctctgcttcgccctctcatttcctgtgagataaaggcggagtctctctccagttattttgctcatccatcgattcttaggtatgactcgtttctctcagatctgtgattctttataatctcgtcgttcttcaaatcattgttatattcgtttcttcgatctgtgttttttagatctgtaaggtaaatgagacgtttcgatctgtagatctgattgttatattgatagattatgttatctgctttgcttaaagtccgatcggaatgttttgtgctcattgtcgaatatctgatgtatcggtttcatagatctgcttctttttgtgcgtttcgttgatctgataatcttctagtgatcaaaatcgtttggatctgttgactttagtttaaaatgtatccgatctgatgtcgaggcttcattattggaagttgttattgttgtaatcctgatttaagttgctgttcttaaatttatatgatctttgcgttataatatgacatggtagatcttggttcatggttcactgttttccaataaacttggtttgtttggttggatagcgttctgtgatacgaccatgtcttgtgttggataagaattctctgaatttccttggctggtttgtagtatgttattcacgtctggtttctcatcaatgattatgtgattttgcagggtaccatgctgtccccagcaaccaccttcagcagttcttcttcatctagctcaccctctagggcccatgcacgcgcccctactcgtttcgctgtagccgcctccgctagagcagcacgcttcagacctgcaagagccatggccgcctcagatgacccaaggggagggaggtccgttgcagtggttggagccggcgtaagcggcctcgctgccgcataccgcttgagaaaaagaggtgtacaggtgacagtgttcgaggcagctgaccgtgccggcggtaaaatccgtaccaatagcgaaggaggctttatatgggacgagggtgctaacactatgaccgaaagtgaactggaggcaagcaggctcatagatgatttgggactgcaaggcaagcaacaatatcccaattctcagcataaacgctatattgtaaaggatggcgctcctacactgattcccagcgacccaatagctttgatgaaaagcactgtgctcagcactaaatccaaactgaagctcttccttgaaccatttctctatgagaagagtagccgcagaacatcaggtaaggtgagcgatgaacacctgagcgagtcagtggctagcttcttcgagcgtcactttggcaaggaggtagtggattatctcattgatccatttgtggccggtacctcagggggagatcccgaatccctcagtattcgccacgcatttcctgctctctggaaccttgaaaataagtacggatctgtcatagctggggctattctcagtaagctctccactaagggtgattcagttaagacaggtggtgctagcccagggaaaggtagaaataaacgtgtgtcattcagcttccatgggggaatgcaaagtctgatagatgcattgcataatgaagtgggtgatgggaacgtgaaactcggaacagaggtgctttcacttgcttgctgctgcgatggggtttcttccagcggcggttggtctatctccgtagattcaaaggatgctaaggggaaggacctgcgtaaaaatcagtccttcgacgctgtcataatgaccgcacctttgagtaacgttcaaaggatgaaattcaccaaggggggagtaccctttgtccttgatttcttgcctaaggttgattacctccccctgagtcttatggtaactgcctttaaaaaagaagatgtgaaaaaaccactcgagggtttcggtgctcttataccttataaagaacaacagaaacatgggctgaaaactctcggcaccatgttctccagcatgatgttcccagatagggctccaaacgatcagtatctctacacatctatgatcgggggaagtcataatcgtgaccttgccggggcccccacagccatattgaagcaattggtaactagcgatctcagaaaacttttgggagtcgaaggccaaccaacttttgttaaacatgtgcactggcgtaatgcatttcccctgtatggtcaaaattacgaccttgtactggaagccatcgcaaagatggaaaacaatttgcctggcttcttttacgccgggaacaacaaggacggacttgctgttgggaacgtgatagccagcggttcaaaggccgccgacctggtaatctcctacctggagagttgtaccgatcaggataactagtgaacgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcactagtgaattcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttag

(19) United States
(12) Patent Application Publication
(10) Pub. No.: US 2025/0230462 A1
(43) Pub. Date: Jul. 17, 2025

(54) PPO2 POLYPEPTIDE HAVING TOLERANCE TO PPO INHIBITOR HERBICIDE AND APPLICATION

(71) Applicant: QINGDAO KINGAGROOT CHEMICAL COMPOUND CO., LTD., Qingdao, Shandong (CN)

(72) Inventors: Sudong MO, Qingdao, Shandong (CN); Lei WANG, Qingdao, Shandong (CN); Guizhi LIU, Qingdao, Shandong (CN); Yanmin LUO, Qingdao, Shandong (CN); Bo CHEN, Qingdao, Shandong (CN)

(21) Appl. No.: 18/838,151

(22) PCT Filed: Feb. 18, 2023
(86) PCT No.: PCT/CN2023/077005

§ 371 Date: Apr. 3, 2025

(30) Foreign Application Priority Data
Mar. 29, 2022 (CN) 202210316331.0

(51) Int. Cl. C12N 15/82; C12N 9/02

(52) U.S. Cl. CPC C12N 15/8274; C12N 9/001; C12N 15/8205; C12Y 103/03004

(57) ABSTRACT

The present invention relates to the technical field of biology, and provides a PPO2 polypeptide having tolerance to a PPO inhibitor herbicide and an application. When applied to a plant, the PPO2 polypeptide can greatly improve the resistance of the plant to a PPO inhibitor herbicide. The PPO2 polypeptide can be used for plants including cash crops, and can be selected to be used according to herbicide resistance characteristics and herbicides, thereby achieving the purpose of economically controlling weed growth.

### FIELD OF THE INVENTION

The present invention relates to the field of biotechnology. More specifically, the present invention relates to a PPO2 polypeptide with tolerance to protoporphyrinogen oxidase (PPO) inhibitor herbicides and application.

### BACKGROUND OF THE INVENTION

Weeds are one of the core factors that affect crop yield in agricultural production. Herbicides are the main technical means to control weeds. The Weed Science Society of America (weedscience.org) has divided herbicides into 28 categories based on their mechanism of action according to different target sites in plants. Among them, group 14 (HRAC GROUP E) are inhibitors that inhibit Protoporphyrinogen IX oxidase (http://www.weedscience.org/).

Protoporphyrinogen IX oxidase (PPOX, PPX or PPO; EC 1.3.3.4) is the last common enzyme in the chlorophyll and heme synthesis pathways. Under aerobic conditions, PPO catalyzes the conversion of protoporphyrinogen (Protoporphyrinogen IX) to protoporphyrin (Protoporphyrin IX).

In plants, PPO is an important target site for herbicides. The inhibition of protoporphyrinogen oxidase in plants could lead to an accumulation of protoporphyrinogen which is the substrate of this reaction in cells, and the accumulation of protoporphyrinogen in chloroplasts and mitochondria in cells results in a non-enzymatic oxidation of protoporphyrinogen by O₂. The non-enzymatic oxidation of protoporphyrinogen produces singlet oxygen in the presence of light. Singlet oxygen causes the oxidation of lipids in cell endomembrane systems and leads to the oxidative disintegration of these endomembrane systems, thereby killing plant cells (Future Med Chem. 2014 April; 6 (6): 597-599. doi: 10.4155/fmc.14.29).

The evolutionary relationship of PPO enzymes in PPO organisms was studied by sequence similarity and PPO can be divided into three categories: HemG, HemJ and Hem Y. In most cases, a single species possesses only one of these. Among them, HemG is generally distributed in Gammaproteobacteria, and HemJ is distributed in Alphaproteobacteria, and both are transferred to other proteobacteria and cyanobacteria, while HemY is the only PPO enzyme distributed in eukaryotes (Genome Biol Evol. 2014 August; 6 (8): 2141-55. doi: 10.1093/gbe/evu170).

There are generally at least two types of PPO genes in plants. They are named PPO1 and PPO2, respectively, wherein, PPO1 is generally located in chloroplasts of plants, while PPO2 is generally located in the mitochondria of plant cells. However, the mRNAs of PPO2 genes in some amaranth plants have different translation initiation sites, which can produce PPO2 polypeptide of various lengths. For example, the PPO2 gene in spinach (Spinacia oleracea L) is able to express 26 polypeptides of various lengths, and two PPO2 proteins with molecular weights of about 58 KD and 56 KD, respectively. The longer one is located in the chloroplasts and the shorter one is located in the mitochondria (J Biol Chem. 2001 Jun. 8; 276 (23): 20474-81. doi: 10.1074/jbc.M101140200. Epub 2001 Mar. 23).

When PPO activity is inhibited by a certain compound, the generation of chlorophyll and heme is also be inhibited. The substrate Protoporphyrinogen IX is separated from the normal porphyrin biosynthesis pathway, quickly breaks away from the chloroplast and enters the cytoplasm, oxidized to Protoporphyrin IX which is accumulated on the cell membrane. The accumulated Protoporphyrin IX produces highly active singlet oxygen (102) under the action of light and oxygen molecules which destroys cell membranes and rapidly leads to death of plant cells. Due to the use of PPO herbicides, there have been cases of weeds resistant to specific types of PPO herbicides in the world (Pest Manag Sci. 2014 September; 70 (9): 1358-66. doi: 10.1002/ps.3728. Epub 2014 Feb. 24).

For example, the deletion of glycine at position 210 (AG210) in the PPO2L gene in Amaranthus tuberculatus confers resistance to the herbicide lactofen (Proc Natl Acad Sci USA. 2006 Aug. 15; 103 (33): 12329-34. doi: 10.1073/pnas.0603137103. Epub 2006 Aug. 7).

The mutation of arginine at position 98 into glycine or methionine (R98G, R98M) in the PPO2 gene in Amaranthus palmeri confers resistance to the herbicide fomesafen (Pest Manag Sci. 2017 August; 73 (8): 1559-1563. doi: 10.1002/ps.4581. Epub 2017 May 16).

The mutation of glycine at position 399 into alanine (G399A) in the PPO2 gene in Amaranthus palmeri confers resistance to the herbicide fomesafen (Front Plant Sci. 2019 May 15; 10:568. doi: 10.3389/fpls.2019.00568. eCollection 2019).

The mutation of arginine at position 98 in the PPO2 gene into leucine (R98L) in Ambrosia artemisiifolia confers resistance to the herbicide flumioxazin (Weed Science, 60 (3): 335-344 (2012)).

### BRIEF SUMMARY OF THE INVENTION

The present invention relates to a PPO2 polypeptide which is tolerant to PPO inhibitor herbicides or a bioactive fragment thereof.

The present invention also relates to an isolated polynucleotide, and the corresponding plant genome, vector construct or host cell.

In another aspect, the present invention also provides a production method for plant cells or plants capable of producing or increasing tolerance to protoporphyrinogen oxidase herbicides, and a plant produced by the method.

In yet another aspect, the present invention provides a method for conferring or increasing tolerance to protoporphyrinogen oxidase herbicides in plants.

The present invention also provides a method of producing or increasing tolerance of plant cells, plant tissues, plant parts or plants to protoporphyrinogen oxidase herbicides.

The present invention additionally provides a use of the protein or bioactive fragment thereof, or the polynucleotide for producing or increasing tolerance of host cells, plant cells, plant tissues, plant parts or plants to protoporphyrinogen oxidase herbicides.

The present invention additionally relates to a method for controlling weeds in plant cultivation sites.

### BRIEF DESCRIPTION OF THE SEVERAL VIEWS OF THE DRAWING(S)

FIG. 1 shows the cell growth levels of the PPO-defective E. coli (ΔhemG) transformants transformed with the wild-type OsPPO2 gene (represented as OsPPO2 WT) or various OsPPO2 mutant genes when treated with compound A at concentrations of 0 μM, 5 μM, 50 μM, 100 μM and 200 μM, respectively.

FIG. 2 shows the cell growth levels of the PPO-defective E. coli (ΔhemG) transformants transformed with the various OsPPO2 F442 mutant genes when treated with compound A at concentrations of 0 μM, 5 μM, 20 μM, 50 μM, 100 UM and 200 μM, respectively; wherein, the resistance of OsPPO2 F442M is different from that shown in FIG. 1 due to variations in culture time and so on.

FIG. 3 shows the cell growth levels of the PPO-defective E. coli (ΔhemG) transformants transformed with the wild-type OsPPO2 gene (represented as OsPPO2 WT) or the various OsPPO2 L422 mutant genes when treated with compound A at concentrations of 0 μM and 5 μM, respectively.

FIGS. 4-6 respectively show the Arabidopsis thaliana seeds overexpressing rice PPO2 WT and L422M/F442M treated with compound A, flumioxazin and saflufenacil of different concentrations. Compared with the wild-type Arabidopsis thaliana, overexpression of rice PPO2 WT and L422M/F442M in Arabidopsis thaliana both exhibit certain tolerance to the compound, however, overexpression of L422M/F442M has stronger tolerance to the compound than overexpression of the wild types. Wherein, “wild-type” represents wild-type Arabidopsis thaliana; “pHSE-OsPPO2 WT” represents overexpressing rice PPO2; “pHSE-OsPPO2 L422M F442M” represents overexpressing rice PPO2 L422M/F442M.

FIG. 7 shows the rice seedlings overexpressing rice PPO2 WT and L422M/F442M sprayed with compound A of different concentrations. Compared with the wild-type strain, the rice seedlings overexpressing PPO2 WT and L422M/F442M both exhibit certain tolerance to compound A. Wherein, “WT” represents wild-type Jinjing 818.

FIG. 8 shows the screening results for wild-type and mutant maize and soybean PPO2 genes using compound A.

FIG. 9 shows the schematic of the maize transgenic recombinant vector.

FIG. 10 shows the resistance test to compound A of rice OsPPO2 L422M/F442M overexpressed in maizes.

FIG. 11 shows the schematic of the soybean transgenic recombinant vector.

FIG. 12 shows the resistance test to compound A of rice OsPPO2 L422M/F442M overexpressed in soybeans.

FIG. 13 shows the sequencing peak map of gene editing at the rice PPO2 L422M/F442M site.

FIG. 14 shows the resistance test to compound A of the rice materials edited at the OsPPO2 L422M/F442M site.

| Sequence No. | Name |
|---|---|
| SEQ ID NO: 1 | Amino acid sequence of wild-type rice PPO2 |
| | (OsPPO2 WT) |
| SEQ ID NO: 2 | Amino acid sequence of rice PPO2 mutant (OsPPO2 |
| | L422M) |
| SEQ ID NO: 3 | Amino acid sequence of rice PPO2 mutant (OsPPO2 |
| | F442M) |
| SEQ ID NO: 4 | Amino acid sequence of rice PPO2 mutant (OsPPO2 |
| | L422M/F442M) |
| SEQ ID NO: 5 | Amino acid sequence of wild-type maize PPO2 |
| | (ZmPPO2 WT) |
| SEQ ID NO: 6 | Amino acid sequence of maize PPO2 mutant |
| | (ZmPPO2L413M/F433M) |
| SEQ ID NO: 7 | Amino acid sequence of wild-type soybean PPO2 |
| | (GmPPO2 WT) |
| SEQ ID NO: 8 | Amino acid sequence of soybean PPO2 mutant |
| | (GmPPO2 L370M/F390M) |
| SEQ ID NO: 9 | Nucleotide sequence of rice Act1 promoter |
| SEQ ID NO: 10 | Nucleotide sequence of CTP-MDH |
| SEQ ID NO: 11 | Nucleotide sequence of maize codon optimized |
| | OsPPO2-422-442 |
| SEQ ID NO: 12 | Nucleotide sequence of T-NoS |
| SEQ ID NO: 13 | Nucleotide sequence of P-E35S |
| SEQ ID NO: 14 | Nucleotide sequence of Pat |
| SEQ ID NO: 15 | Nucleotide sequence of CaMV poly (A) signal |
| SEQ ID NO: 16 | Nucleotide sequence of P-CsVMV |
| SEQ ID NO: 17 | Nucleotide sequence of Pat |
| SEQ ID NO: 18 | Nucleotide sequence of T-E9 |
| SEQ ID NO: 19 | Nucleotide sequence of P-AtNt1 |
| SEQ ID NO: 20 | Nucleotide sequence of soybean codon optimized |
| | OsPPO2-422-442-Gm1 |
| SEQ ID NO: 21 | Nucleotide sequence of T-Nos |
| SEQ ID NO: 22 | Complete sequence of maize transgenic vector |
| SEQ ID NO: 23 | Complete sequence of soybean transgenic vector |

### DETAILED DESCRIPTION OF THE INVENTION

Some terms used in this specification are defined as follows.

The “herbicide” in the present invention refers to an active ingredient capable of killing or controlling or detrimentally transforming the growth of plants. The “herbicide tolerance” or “herbicide resistance” in the present invention refers to that even after treatment of a herbicide which can kill ordinary or wild-type plants or inhibit growth thereof, or weaken or eliminate the growth ability of a plant compared with that of wild-type plants, the plant continues to grow. The above-mentioned herbicides include protoporphyrinogen oxidase (PPO) inhibitor herbicides. The PPO inhibitor herbicides may be classified into pyrimidinediones, diphenyl-ethers, phenylpyrazoles, N-phenylphthalimides, thiadiazoles, oxadiazoles, triazolinones, oxazolidinediones and other herbicides of different chemical structures.

In general, if the PPO inhibiting herbicides and/or other herbicidal compounds that can be used in the context of the present invention as described herein are able to form geometric isomers such as E/Z isomers, it is then possible to use pure isomers of both and mixtures thereof in the compositions according to the present invention. If the PPO inhibiting herbicides and/or other herbicidal compounds as described herein have one or more chiral centers and therefore exist as enantiomers or diastereoisomers, it is then possible to use both, pure enantiomers and diastereoisomers, and mixtures thereof, in the compositions according to the present invention. If the PPO inhibiting herbicides and/or other herbicidal compounds as described herein have ionizable functional groups, they may also be used in the form of agriculturally acceptable salts thereof. In general, the salts of those cations and the acid addition salts of those acids are suitable, the cations and anions of which have no adverse effect on the activity of the active compounds, respectively. Preferred cations are the ions of alkali metals, preferably lithium, sodium and potassium; ions of alkaline earth metals, preferably calcium and magnesium; and ions of transition metals, preferably manganese, copper, zinc and iron; further ammonium and substituted ammonium, wherein 1 to 4 of the hydrogen atoms are replaced by C₁-C₄-alkyl, hydroxyl-C₁-C₄-alkyl, C₁-C₄-alkoxy-C₁-C₄-alkyl, hydroxyl-C₁-C₄-alkoxy-C₁-C₄-alkyl, phenyl or benzyl, preferably ammonium, methylammonium, isopropylammonium, dimethylammonium, diisopropylammonium, trimethylammonium, heptylammonium, dodecylammonium, tetradecylammonium, tetramethylammonium, tetraethylammonium, tetrabutylammonium, 2-hydroxyethylammonium (olamine salt), 2-(2-hydroxyeth-1-oxy) eth-1-yl ammonium (diglycolamine salt), di(2-hydroxyeth-1-yl) ammonium (diolamine salt), tri (2-hydroxyethyl) ammonium (trinitroethanolamine salt), tri (2-hydroxypropyl) ammonium, benzyltrimethylammonium, benzyltriethylammonium, N,N,N-trimethylethanolammonium (choline salt); in addition, phosphonium ions, sulfonium ions, preferably tri (C₁-C₄-alkyl) sulfonium such as trimethylsulfonium, and sulfoxonium ions, preferably tri (C₁-C₄-alkyl) sulfoxonium ions, and eventually polyamines such as salts of N,N-bis-(3-aminopropyl)methylamine and diethylenetriamine. The available anions of acid addition salts are mainly chloride, bromide, fluoride, iodide, bisulfate, methyl sulfate, sulfate, dihydrogen phosphate, hydrogen phosphate, nitrate, bicarbonate, carbonate, hexafluorosilicate, hexafluorophosphate, benzoate and also anions of C₁-C₄-alkanoic acids, preferably formate, acetate, propionate and butyrate.

The PPO inhibiting herbicides and/or other herbicidal compounds having a carboxyl as described herein may be used in the form of acids, agriculturally suitable salts as mentioned above, or otherwise agriculturally acceptable derivatives, for example, as amides such as mono- and di-C₁-C₆ alkyl amides or arylamides; as esters such as allyl esters, propargyl esters, C₁-C₁₀ alkyl esters, alkoxyalkyl esters, tefuryl ((tetrahydrofuran-2-yl)methyl) esters and also as thioesters such as C₁-C₁₀ alkyl thioesters. Preferred mono- and di-C₁-C₆ alkyl amides are methyl- and di-methylamide. Preferred arylamides are, for example, N-anilide and 2-chloroanilide. Preferred alkyl esters are, for example, methyl, ethyl, propyl, isopropyl, butyl, isobutyl, pentyl, mexyl (1-methylhexyl), meptyl (1-methylheptyl), heptyl, octyl or isooctyl (2-ethylhexyl) esters. Preferred C₁-C₄ alkoxy-C₁-C₄-alkyl esters are linear or branched C₁-C₄ alkoxyethyl esters such as 2-methoxylethyl esters, 2-ethoxylethyl esters, 2-butoxyethtyl (butotyl) esters, 2-butoxypropyl esters or 3-butoxypropyl esters. An example of linear or branched C₁-C₁₀ alkyl thioesters is ethyl thioester.

In an exemplary embodiment, pyrimidinedione herbicides include, but are not limited to, butafenacil (CAS NO: 134605-64-4), saflufenacil (CAS NO: 372137-35-4), benzfendizone (CAS NO: 158755-95-4), tiafenacil (CAS NO: 1220411-29-9), [3-[2-chloro-4-fluoro-5-(1-methyl-6-trifluoromethyl-2,4-dioxo-1,2,3,4-tetrahydropyrimidin-3-yl) phenoxy]-2-pyridyloxy] ethyl acetate (epyrifenacil, CAS NO: 353292-31-6), 1-methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS NO: 1304113-05-0), 3-[7-chloro-5-fluoro-2-(trifluoromethyl)-1H-benzimidazol-4-yl]-1-methyl-6-(trifluoromethyl)-1H-pyrimidine-2,4-dione (CAS NO: 212754-02-4), flupropacil (CAS NO: 120890-70-2), uracils containing isoxazoline

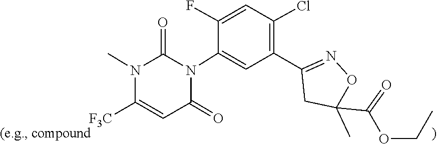 [SMILES: CCOC(=O)C1(C)CC(c2cc(-n3c(=O)cc(C(F)(F)F)n(C)c3=O)c(F)cc2Cl)=NO1]

disclosed in CN105753853A, uracil pyridines disclosed in WO2017/202768 and uracils disclosed in WO2018/019842.

Diphenyl-ether herbicides include, but are not limited to, fomesafen (CAS NO: 72178-02-0), oxyfluorfen (CAS NO: 42874-03-3), aclonifen (CAS NO: 74070-46-5), lactofen (CAS NO: 77501-63-4), chlomethoxyfen (CAS NO: 32861-85-1), chlornitrofen (CAS NO: 1836-77-7), fluoroglycofen-ethyl (CAS NO: 77501-90-7), acifluorfen or acifluorfen-sodium (CAS NO: 50594-66-6 or 62476-59-9), bifenox (CAS NO: 42576-02-3), ethoxyfen (CAS NO: 188634-90-4), ethoxyfen-ethyl (CAS NO: 131086-42-5), fluoronitrofen (CAS NO: 13738-63-1), furyloxyfen (CAS NO: 80020-41-3), nitrofluorfen (CAS NO: 42874-01-1) and halosafen (CAS NO: 77227-69-1).

Phenylpyrazole herbicides include, but are not limited to, pyraflufen-ethyl (CAS NO: 129630-19-9) and fluazolate (CAS NO: 174514-07-9).

N-phenylphthalimide herbicides include, but are not limited to, flumioxazin (CAS NO: 103361-09-7), cinidon-ethyl (CAS NO: 142891-20-1), flumipropyn (CAS NO: 84478-52-4) and flumiclorac-pentyl (CAS NO: 87546-18-7).

Thiadiazole herbicides include, but are not limited to, fluthiacet-methyl (CAS NO: 117337-19-6), fluthiacet (CAS NO: 149253-65-6) and thidiazimin (CAS NO: 123249-43-4).

Oxadiazole herbicides include, but are not limited to, oxadiargyl (CAS NO: 39807-15-3) and oxadiazon (CAS NO: 19666-30-9).

Triazolinone herbicides include, but are not limited to, carfentrazone (CAS NO: 128621-72-7), carfentrazone-ethyl (CAS NO: 128639-02-1), sulfentrazone (CAS NO: 122836-35-5), azafenidin (CAS NO: 68049-83-2) and bencarbazone (CAS NO: 173980-17-1).

Oxazolidinedione herbicides include, but are not limited to, pentoxazone (CAS NO: 110956-75-7).

Other herbicides include, but are not limited to, pyraclonil (CAS NO: 158353-15-2), flufenpyr-ethyl (CAS NO: 188489-07-8), profluazol (CAS NO: 190314-43-3), trifludimoxazin (CAS NO: 1258836-72-4), N-ethyl-3-(2,6-dichloro-4-trifluoromethylphenoxy)-5-methyl-1H-pyrazole-1-carboxamide (CAS NO: 452098-92-9), N-tetrahydrofurfuryl-3-(2,6-dichloro-4-trifluoromethylphenoxy)-5-methyl-1H-pyrazole-1-carboxamide (CAS NO: 915396-43-9), N-ethyl-3-(2-chloro-6-fluoro-4-trifluoromethylphenoxy)-5-methyl-1H-pyrazole-1-carboxamide (CAS NO: 452099 May 7), N-tetrahydrofurfuryl-3-(2-chloro-6-fluoro-4-trifluoromethylphenoxy)-5-methyl-1H-pyrazole-1-carboxamide (CAS NO: 452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (CAS NO: 451484-50-7), 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione (CAS NO: 1300118-96-0), Methyl (E)-4-[2-chloro-5-[4-chloro-5-(difluoromethoxy)-1H-methyl-pyrazol-3-yl]-4-fluoro-phenoxy]-3-methoxy-but-2-enoate (CAS NO: 948893-00-3), phenylpyridines disclosed in WO2016/120116, benzoxazinone derivatives disclosed in EP09163242.2 and the compounds represented by Formula I

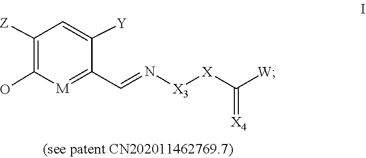

In another exemplary embodiment, Q represents

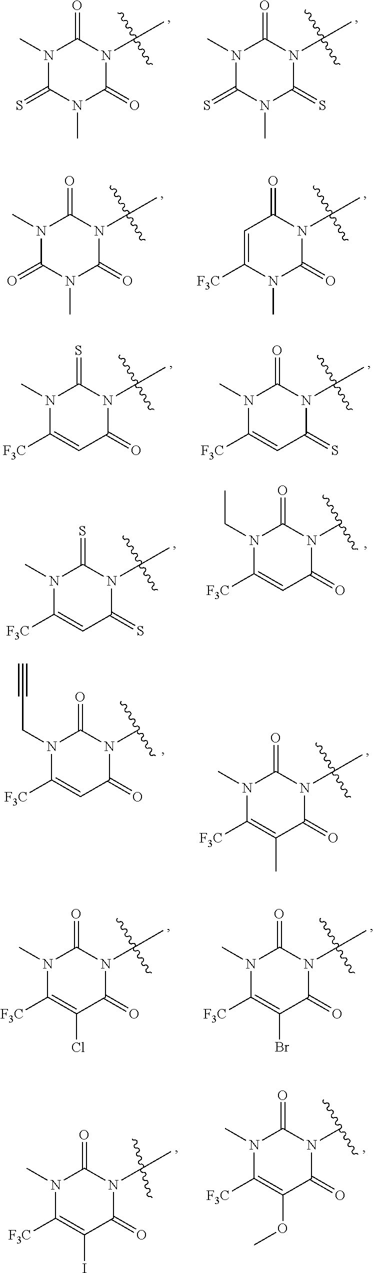

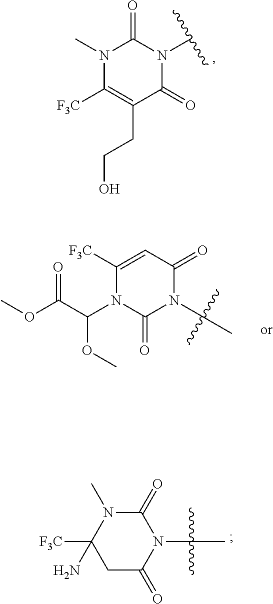 [SMILES: *n1c(=O)c(CCO)c(C(F)(F)F)n(C)c1=O ; *n1c(=O)cc(C(F)(F)F)n(C(OC)C(=O)OC)c1=O ; *N1C(=O)CC(N)(C(F)(F)F)N(C)C1=O]

Y represents halogen, halo C₁-C₆ alkyl or cyano;
Z represents halogen;
M represents CH or N;
X represents —CX₁X₂—(C₁-C₆ alkyl)ₙ-, —(C₁-C₆ alkyl)-CX₁X₂—(C1-C6 alkyl)ₙ- or —(CH₂)ᵣ—, n represents 0 or 1, r represents an integer of 2 and above;
X₁ and X₂ independently represent hydrogen, halogen, C1-C6 alkyl, C2-C6 alkenyl, C2-C6 alkynyl, halo C1-C6 alkyl, halo C2-C6 alkenyl, halo C2-C6 alkynyl, C3-C6 cycloalkyl, C3-C6 cycloalkyl C1-C6 alkyl, C1-C6 alkoxy, C1-C6 alkylthio, hydroxyl C1-C6 alkyl, C1-C6 alkoxy C1-C6 alkyl, phenyl or benzyl, respectively;
X₃ and X₄ independently represent O or S, respectively;
W represents hydroxyl, C1-C6 alkoxy, C2-C6 alkenoxy, C2-C6 alkynoxy, halo C1-C6 alkoxy, halo C2-C6 alkenoxy, halo C2-C6 alkynoxy, C3-C6 cycloalkoxy, phenoxy, mercapto, C1-C6 alkylthio, C2-C6 alkenylthio, C2-C6 alkynylthio, halo C1-C6 alkylthio, halo C2-C6 alkenylthio, halo C2-C6 alkynylthio, C3-C6 cycloalkylthio, phenylthio, amino or C1-C6 alkylamino.

In another exemplary embodiment, the compound represented by Formula I is selected from compound A: Q represents

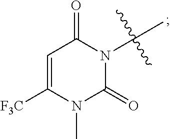 [SMILES: *n1c(=O)cc(C(F)(F)F)n(C)c1=O]

Y represents chlorine; Z represents fluorine; M represents CH; X represents —C*X₁X₂—(C1-C6 alkyl)ₙ- (C* is the chiral center in R configuration), n represents 0; X₁ represents hydrogen; X₂ represents methyl; X₃ and X₄ independently represent O; W represents methoxy.

The PPO inhibiting herbicides described above which are useful in practicing the present invention are generally best applied in combination with one or more other herbicides to obtain control of a variety of undesired plants. For example, PPO inhibiting herbicides can also be used in combination with additional herbicides to which crop plants are naturally tolerant or made resistant through expression of one or more additional transgenes as previously described. When used in combination with other targeting herbicides, the claimed compounds of the present invention may be formulated with one or more other herbicides, tank-mixed with one or more other herbicides, or applied sequentially with one or more other herbicides.

Components of a suitable mixture are, for example, selected from herbicides of categories b1) to b15):
b1) lipid biosynthesis inhibitors;
b2) acetolactate synthase inhibitors (ALS inhibitors);
b3) photosynthesis inhibitors;
b4) Protoporphyrinogen IX oxidase inhibitors;
b5) bleaching herbicides;
b6) 5-enolpyruvylshikimate-3-phosphate synthase inhibitors (EPSP inhibitors);
b7) glutamine synthase inhibitors;
b8) 7,8-dihydropteroate synthase inhibitors (DHP inhibitors);
b9) mitotic inhibitors;
b10) very long chain fatty acid synthesis inhibitors (VLCFA inhibitors);
b11) cellulose biosynthesis inhibitors;
b12) decoupler herbicides;
b13) auxinic herbicides;
b14) auxin transport inhibitors; and
b15) other herbicides selected from bromobutide, chlorflurenol, chlorflurenol-methyl, cinmethylin, cumyluron, dalapon, dazomet, difenzoquat, difenzoquat-metilsulfate, dimethipin, DSMA, dymron, endothal and salts thereof, etobenzanid, flamprop, flamprop-isopropyl, flamprop-methyl, flamprop-M-isopropyl, flamprop-M-methyl, flurenol, flurenol-butyl, flurprimidol, fosamine, fosamine-ammonium, indanofan, indaziflam, maleic hydrazide, mefluidide, metam, methiozolin (CAS NO: 403640-27-7), methyl azide, methyl bromide, methyl-dymron, methyl iodide, MSMA, oleic acid, oxaziclomefone, pelargonic acid, pyributicarb, quinoclamine, triaziflam, ridiphane and 6-chloro-3-(2-cyclopropyl-6-methylphenoxy)-4-pyridazinol (CAS NO: 499223-49-3) and salts and esters thereof;
and agriculturally acceptable salts or derivatives thereof.

In addition, when used in combination with the other herbicidal compounds as described above, it may be useful to apply PPO inhibiting herbicides in combination with safeners. Safeners are compounds that prevent or reduce the damage to useful plants without having a significant impact on the herbicidal action of herbicides on undesired plants. They can be applied before sowing (e.g., during seed treatment, on shoots or seedlings), or before or after the germination of useful plants.

Moreover, safeners, PPO inhibiting herbicides and/or other herbicidal compounds may be applied simultaneously or sequentially.

PPO inhibiting herbicides and herbicidal compounds of categories b1)-b15) and safeners are known herbicides and safeners, for example, see WO2013/189984; The Compendium of Pesticide Common Names (http://www.alanwood.net/pesticides/); Farm Chemicals Handbook 2000, Vol. 86, Meister Publishing Company, 2000; B. Hock, C. Fedtke, R. R. Schmidt, Herbizide [Herbicide], Georg Thieme Verlag, Stuttgart, 1995; W. H. Ahrens, Herbicide Handbook, 7th edition, Weed Science Society of America, 1994 and K. K. Hatzios, Herbicide Handbook, 7th edition supplement, Weed Science Society of America, 1998.

The term “weed control” is to be understood as killing weeds and/or delaying or inhibiting normal growth of weeds. In the broadest sense, weeds are understood as all plants known to grow in locations where they are not desired, e.g., (crop) plant cultivation sites. Weeds of the present invention include, for example, dicotyledonous or monocotyledonous weeds. Dicotyledonous weeds include, but are not limited to, the following genera of weeds: Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus and Taraxacum. Monocotyledonous weeds include, but are not limited to, the following genera of weeds: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus and Apera. Furthermore, the weeds of the present invention may include, for example, crop plants that are growing in an undesired location. For instance, if maize plants are unwanted in a field of soybean plants, a volunteer maize plant present in a field primarily comprising soybean plants can be considered as a weed.

The term “plant” is used in its broadest sense, as it relates to organic matters and is intended to encompass eukaryotic organisms that are members of the kingdom Plantae, examples of which include, but are not limited to, a vascular plant, vegetable, grain, flower, tree, herb, bush, grass, vine, fern, moss, fungus and algae, etc., as well as a clone, offset and plant part for asexual propagation (e.g., a cutting, piping, shoot, rhizome, underground stem, clump, crown, bulb, corm, tuber, rhizome, plant/tissue produced in tissue cultures, etc.). The term “plant” further encompasses a whole plant, ancestor and progeny of a plant and plant part, including a seed, shoot, stem, leaf, root (including tuber), flower, floret, fruit, pedicle, peduncle, stamen, anther, stigma, style, ovary, petal, sepal, carpel, root tip, root cap, root hair, leaf hair, seed hair, pollen grain, microspore, cotyledon, hypocotyl, epicotyl, xylem, phloem, parenchyma, endosperm, companion cell, guard cell and any other know organ, tissue and cell of a plant, and a tissue and organ each comprising the target gene/nucleic acid. The term “plant” also encompasses a plant cell, suspension culture, callus, embryo, meristematic region, gametophyte, sporophyte, pollen and microspore, again wherein each of the foregoing comprises the target gene/nucleic acid.

Plants that are particularly useful in the methods of the invention include all plants which belong to the superfamily Viridiplantae, in particular monocotyledonous and dicotyledonous plants, including fodder or forage legumes, ornamental plants, food crops, trees or bushes selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp., Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp.), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybermuim, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugar cane, sunflower, tomato, squash, tea and algae, amongst others. According to preferred embodiments of the present invention, the plants are crop plants. Examples of crop plants include particularly soybean, sunflower, canola, alfalfa, rapeseed, cotton, tomato, potato or tobacco. Further preferably, the plants are monocotyledonous plants, such as sugarcane. Further preferably, the plants are cereals, such as rice, maize, wheat, barley, millet, rye, sorghum or oats.

In the present invention, the term “plant tissue” or “plant part” includes a plant cell, protoplast, plant tissue culture, plant callus, plant piece as well as a plant embryo, pollen, ovule, seed, leaf, stem, flower, branch, shoot, fruit, pit, ears, root, root tip, anther, etc.

In the present invention, the “plant cell” is to be understood as any cell from or found in a plant, which is capable of forming, for example, an undifferentiated tissue such as a callus, differentiated tissue such as an embryo, plant component, plant or seed.

In the present invention, the “host organism” is to be understood as any unicellular or multicellular organism into which a mutant protein-coding nucleic acid can be introduced, including, for example, a bacterium such as E. coli, fungus such as a yeast (e.g., Saccharomyces cerevisiae), mold (e.g., Aspergillus), plant cell and plant, etc.

In one aspect, the present invention provides a PPO2 polypeptide or a bioactive fragment thereof which is tolerant to PPO inhibitor herbicides, comprising an amino acid sequence having/having only the following mutation(s) compared with the amino acid sequence as set forth in SEQ ID NO: 1: the amino acid corresponding to position 422 in the amino acid sequence set forth in SEQ ID NO: 1 is mutated from leucine into methionine, and/or the amino acid at position 442 is mutated from phenylalanine into methionine;
comprising an amino acid sequence having/having only the following mutation(s) compared with the amino acid sequence as set forth in SEQ ID NO: 5: the amino acid corresponding to position 413 in the amino acid sequence set forth in SEQ ID NO: 5 is mutated from leucine into methionine, and/or the amino acid at position 433 is mutated from phenylalanine into methionine; or,
comprising an amino acid sequence having/having only the following mutation(s) compared with the amino acid sequence as set forth in SEQ ID NO: 7: the amino acid corresponding to position 370 in the amino acid sequence set forth in SEQ ID NO: 7 is mutated from leucine into methionine, and/or the amino acid at position 390 is mutated from phenylalanine into methionine.

In one embodiment, the amino acid sequence further has at least 80%, at least 85%, at least 90%, at least 95%, at least 96%, at least 97%, at least 98% or at least 99% sequence identity to the amino acid sequences set forth in SEQ ID NO: 1, 5 or 7, respectively.

In another embodiment, the PPO2 polypeptide or bioactive fragment thereof comprises an amino acid sequence that has at least 92%, at least 95%, at least 96%, at least 97%, at least 98%, at least 99% or 100% sequence identity to an amino acid sequence selected from the group consisting of SEQ ID NO: 2, 3, 4, 6 or 8. Preferably, the amino acid sequence of the polypeptide is as set forth in any one of SEQ ID NO: 2, 3, 4, 6 and 8.

The terms “protein”, “polypeptide” and “peptide” can be used interchangeably in the present invention and refer to a polymer of amino acid residues, including a polymer of chemical analogs in which one or more amino acid residues are natural amino acid residues. The proteins and polypeptides of the present invention may be recombinantly produced or chemically synthesized.

For the terms related to amino acid substitution used in the specification, the first letter represents a naturally occurring amino acid at a certain position in a specific sequence; the following number represents the position relative to SEQ ID NO: 1; and the second letter represents a different amino acid that substitutes the natural amino acid. For instance, L422M represents that, relative to the amino acid sequence of SEQ ID NO: 1, the leucine at position 422 is substituted by methionine. For double or multiple mutations, mutations are separated by “/”. For example, L422M/F442M represents that, relative to the amino acid sequence of SEQ ID NO: 1, the leucine at position 422 is substituted by methionine, and the phenylalanine at position 442 is substituted by methionine. Both mutations are present in the specific mutant OsPPO2 protein.

The specific amino acid positions (numbering) in the protein of the present invention are determined by aligning the amino acid sequence of the target protein with SEQ ID NO: 1 and so on, using standard sequence alignment tools. For example, two sequences are aligned using the Smith-Waterman algorithm or the ClustalW2 algorithm, wherein the sequences are considered aligned when the alignment score is the highest. Alignment scores can be calculated according to the method described in Wilbur, W. J. and Lipman, D. J. (1983) Rapid similarity searches of nucleic acid and protein data banks. Proc. Natl. Acad. Sci. USA, 80:726-730. Default parameters are preferably used in the ClustalW2 (1.82) algorithm: Protein gap opening penalty=10.0; Protein gap extension penalty=0.2; Protein weight matrix=Gonnet; Protein/DNA endgap=−1; Protein/DNA GAPDIST=4.

The AlignX program (a part of the Vector NTI WorkGroup) is preferably adopted to accommodate default parameters of multiple alignment (Gap opening penalty: 10; Gap extension penalty: 0.05). The position of a specific amino acid in the protein of the present invention is determined by aligning the amino acid sequence of the protein with SEQ ID NO: 1.

Amino acid sequence identity can be determined by conventional methods using the BLAST algorithm (Altschul et al., 1990, Mol. Biol. 215:403-10) obtained from the National Center for Biotechnology Information, USA (www.ncbi.nlm.nih.gov/) with default parameters.

It is also clear to those skilled in the art that the structure of a protein may be altered without adversely affecting its activity and functionality. For example, one or more conservative amino acid substitutions can be introduced into the amino acid sequence of a protein without adversely affecting the activity and/or three-dimensional configuration of the protein molecule. Examples and embodiments of conservative amino acid substitutions are apparent to those skilled in the art. Specifically, an amino acid residue can be substituted with another amino acid residue that belongs to the same group as the site to be substituted does, that is, using a non-polar amino acid residue to substitute another non-polar amino acid residue; using a polar uncharged amino acid residue to substitute another polar uncharged amino acid, using an alkaline amino acid residue to substitute another alkaline amino acid residue, and using an acidic amino acid residue to substitute another acidic amino acid residue. Conservative substitutions that one amino acid is substituted with another amino acid of the same group are within the scope of the present invention as long as the substitution(s) does not impair the biological activity of the protein.

Therefore, in addition to the above-mentioned mutations, the mutant protein of the present invention may further comprise one or more other mutations, such as conservative substitutions, in the amino acid sequence. Moreover, the present invention also encompasses a mutant protein that also comprises one or more other non-conservative substitutions, as long as the non-conservative substitution(s) does not significantly affect the desired functions or biological activity of the protein of the present invention.

As well known in the art, one or more amino acid residues can be deleted from the N- and/or C-terminus of a protein while the protein still retains its functional activity. Thus, in another aspect, the present invention also relates to fragments that have one or more amino acid residues deleted from N- and/or C-termini of mutant proteins while retain their desired functional activity, which are also within the scope of the present invention and called bioactive fragments. In the present invention, the “bioactive fragment” refers to a portion of the mutant protein of the present invention which retains the biological activity of the mutant protein of the present invention. For example, a bioactive fragment of a mutant protein may be a portion that has one or more (e.g., 1-50, 1-25, 1-10 or 1-5; e.g., 1, 2, 3, 4 or 5) amino acid residues deleted from the N- and/or C-terminus of the protein while still retains the biological activity of the full-length protein.

The term “mutation” refers to a single amino acid variation in a polypeptide and/or at least a single nucleotide variation in a nucleic acid sequence relative to a canonical sequence, wild-type sequence or reference sequence. In some embodiments, the mutation refers to a single amino acid variation in a polypeptide and/or at least a single nucleotide variation in a nucleic acid sequence relative to the nucleotide or amino acid sequence of a PPO protein that is not herbicide-resistant. In some embodiments, the mutation refers to one or more variations at the amino acid site relative to the reference amino acid sequence of PPO2 as set forth in SEQ ID NO: 1, or at the homologous site in a homologous gene of a different species. In some embodiments, mutations may include substitutions, deletions, inversions or insertions. In some embodiments, substitutions, deletions, insertions, or inversions may include 1, 2, 3, 4, 5, 6, 7, 8, 9, 10, 11, 12, 13, 14, 15, 16, 17, 18, 19, 20, 21, 22, 23 or 24 nucleotide variations. In some embodiments, substitutions, deletions, insertions, or inversions may include variations at 1, 2, 3, 4, 5, 6, 7 or 8 amino acid sites.

The term “wild-type”, relative to “mutant”, refers to a phenotype of the highest frequency in a particular population, or a system, organism and gene with this phenotype. In some examples, a wild-type allele refers to a standard allele at a locus, or an allele of the highest frequency in a particular population, which can be represented by a particular amino acid or nucleic acid sequence. For example, the wild-type rice PPO2 protein can be represented by SEQ ID NO: 1; the wild-type maize PPO2 protein can be represented by SEQ ID NO: 5; the wild-type soybean PPO2 protein can be represented by SEQ ID NO: 7.

In another aspect, the present invention also provides an isolated polynucleotide comprising a nucleic acid sequence selected from:
(1) a nucleic acid sequence encoding the PPO2 polypeptide or its bioactive fragment, or a partial sequence or complementary sequence thereof;
(2) a nucleic acid sequence set forth in SEQ ID NO: 11 or 20, or a complementary sequence thereof;
(3) a nucleic acid sequence that hybridizes to the sequence shown in (1) or (2) under stringent conditions; and/or
(4) a nucleic acid sequence encoding the same amino acid sequence as the sequence shown in (1) or (2) due to degeneracy of the genetic code, or a complementary sequence thereof. In one embodiment, the polynucleotide is a DNA molecule.

The terms “polynucleotide”, “nucleic acid”, “nucleic acid molecule” or “nucleic acid sequence” can be used interchangeably, referring to an oligonucleotide, nucleotide or polynucleotide and a fragment or part thereof, which may be single-stranded or double-stranded, representing a sense or antisense strand. Nucleic acids include DNA, RNA or hybrids thereof, and can be of natural or synthetic origin. For example, nucleic acids may include mRNA or cDNA. Nucleic acids may include acids that have been amplified (e.g., by the polymerase chain reaction). The one-letter symbols for nucleotides are as set forth in Appendix A, Chapter 2422 of the Manual of Patent Examining Procedure by the U.S. Patent and Trademark Office (USPTO). In this regard, the nucleotide designation “R” means purine such as guanine or adenine; “Y” means pyrimidine such as cytosine or thymine (uracil in the case of RNA); “M” means adenine or cytosine; “K” means guanine or thymine; and “W” means adenine or thymine. The term “isolated”, when referring to a nucleic acid, means a nucleic acid that is apart from a substantial portion of the genome in which it naturally occurs and/or is substantially separated from other cellular components which naturally accompany the nucleic acid. For example, any nucleic acid that has been produced synthetically (e.g., by serial base condensation) is considered to be isolated. Likewise, a nucleic acid that is recombinantly expressed, or cloned, or produced by a primer extension reaction (e.g., PCR), or otherwise excised from a genome is also considered to be isolated.

It will be apparent to those skilled in the art that a variety of different nucleic acid sequences can encode the amino acid sequence disclosed herein due to the degeneracy of the genetic code. It is within the ability of one of ordinary skill in the art to generate other nucleic acid sequences encoding the same protein, and thus the present invention encompasses nucleic acid sequences that encode the same amino acid sequence due to the degeneracy of the genetic code. For example, in order to achieve high expression of a heterologous gene in a target host organism, such as a plant, the gene can be optimized using host-preferred codons for better expression.

The present invention also provides a plant genome comprising the polynucleotide.

In one embodiment, the plant genome is modified with at least one mutation. In another embodiment, the plant genome is modified with at least two mutations.

The present invention also provides a vector construct comprising the polynucleotide and a homologous or a non-homologous promoter operably linked thereto.

The present invention also provides a vector construct comprising:
(1) a gene of which the nucleotide sequence is as set forth in SEQ ID NO: 11 and a gene of which the nucleotide sequence is set forth in SEQ ID NO: 14;
(2) a gene of which the nucleotide sequence is as set forth in SEQ ID NO: 17 and a gene of which the nucleotide sequence is set forth in SEQ ID NO: 20;
(3) two expression cassettes in tandem, wherein one expression cassette comprises a rice Act1 promoter of which the nucleotide sequence is as set forth in SEQ ID NO: 9, a CTP-MDH chloroplast-localized peptide of which the nucleotide sequence is as set forth in SEQ ID NO: 10, a gene of which the nucleotide sequence is as set forth in SEQ ID NO: 11, and a T-NOS terminator of which the nucleotide sequence is as set forth in SEQ ID NO: 12; the other expression cassette comprises a P-E35S promoter of which the nucleotide sequence is as set forth in SEQ ID NO: 13, a gene of which the nucleotide sequence is as set forth in SEQ ID NO: 14, and a CaMV poly (A) signal terminator of which the nucleotide sequence is as set forth in SEQ ID NO: 15; or,
(4) two expression cassette in tandem, wherein one expression cassette comprises a P-CsVMV promoter of which the nucleotide sequence is as set forth in SEQ ID NO: 16, a gene of which the nucleotide sequence is as set forth in SEQ ID NO: 17, and a T-E9 terminator of which the nucleotide sequence is as set forth in SEQ ID NO: 18; the other expression cassette comprises a P-AtNt1 promoter of which the nucleotide sequence is as set forth in SEQ ID NO: 19, a gene of which the nucleotide sequence is as set forth in SEQ ID NO: 20, and a T-Nos terminator of which the nucleotide sequence is as set forth in SEQ ID NO: 21.

In one specific embodiment, the nucleotide sequence of the vector construct is as set forth in SEQ ID NO: 22 or SEQ ID NO: 23.

The present invention also provides a host cell comprising the polynucleotide or vector construct.

In one embodiment, the host cell is a plant cell.

The present invention also provides a production method for plant cells capable of producing or increasing tolerance to protoporphyrinogen oxidase inhibitor herbicides, which includes using the gene editing approach to produce the above-mentioned polynucleotide in plant cells or using the transgenic approach to introduce the polynucleotide or vector construct described above into plant cells.

The present invention also provides a production method for plants capable of producing or increasing tolerance to protoporphyrinogen oxidase inhibitor herbicides, which includes regenerating the above-mentioned plant cells or the plant cells produced by the above-mentioned method into plants.

The present invention also provides a plant produced by the above-mentioned method.

In one embodiment, the above-mentioned plants or plant cells are non-transgenic.

In another embodiment, the above-mentioned plants or plant cells are transgenic.

The term “transgenic” plant refers to a plant comprising a heterologous polynucleotide. Preferably, a heterologous polynucleotide is stably integrated into a genome, allowing the polynucleotide to be passed on to successive generations. The heterologous polynucleotide can be integrated into a genome alone or as a part of a recombinant expression cassette. The “transgenic” is used herein to refer to any cell, cell line, callus, tissue, plant part or plant whose genotype is altered due to the presence of a heterologous nucleic acid, including those originally altered transgenic organisms or cells, as well as those resulting from crossing or asexual propagation of originally altered transgenic organisms or cells. The term “transgenic” as used herein is not intended to include altering genomes (chromosomal or extrachromosomal) by conventional plant breeding methods (e.g., crossing) or by naturally occurring events (e.g., self-fertilization, random cross-fertilization, non-recombinant viral infection, non-recombinant bacterial transformation, non-recombinant transposition or spontaneous mutation).

The term “gene-edited plant”, “gene-edited plant part” or “gene-edited plant cell” refers to a plant, a part or cell thereof comprising one or more endogenous genes edited by a gene editing system. The “gene editing system” refers to a protein, nucleic acid or a combination thereof capable of modifying a target locus in an endogenous DNA sequence when introduced into a cell. Many gene editing systems suitable for use in the methods of the present invention are known in the art, including but not limited to the zinc finger nucleases (ZFNs) system, transcription activator-like effector nuclease (TALEN) system and CRISPR/Cas system. The term “gene editing” as used herein generally refers to a technique for carrying out DNA insertions, deletions, alterations or substitutions in a genome. For example, the gene editing may include the knock-in approach. The knock-in approach can be a method of operation commonly used by those skilled in the art, for example, see “Gene Targeting: A Practical Approach”, Joyner A, editor. Oxford, U.K.: Oxford Univ. Press; (2000).

The present invention also provides a method for conferring or increasing tolerance to protoporphyrinogen oxidase herbicides in plants, which includes introducing a modification into a gene that encodes a protein having protoporphyrinogen oxidase activity to produce the PPO2 polypeptide or bioactive fragment thereof.

The present invention also provides a method of producing or increasing tolerance of plant cells, plant tissues, plant parts or plants to protoporphyrinogen oxidase herbicides, comprising expressing the PPO2 polypeptide or bioactive fragment thereof in the plant cells, plant tissues, plant parts or plants;
or, comprising crossing a plant that expresses the PPO2 polypeptide or bioactive fragment thereof with another plant, as well as screening plants or parts thereof that are capable of producing or increasing tolerance to protoporphyrinogen oxidase herbicides;
or, comprising performing gene editing on a protein that has protoporphyrinogen oxidase activity of the plant cells, plant tissues, plant parts or plants, so as to express the PPO2 polypeptide or its bioactive fragment therein.

The present invention also provides a use of the PPO2 polypeptide or bioactive fragment thereof, or the polynucleotide for producing or increasing tolerance of host cells, plant cells, plant tissues, plant parts or plants to protoporphyrinogen oxidase herbicides.

In one embodiment, the host cell is a bacterial cell or fungal cell.

The above-mentioned herbicide-resistant PPO protein is obtained through the most common natural extraction and refining methods in the art. It can also be obtained as a synthetic protein through chemical synthesis methods, or as a recombinant protein through genetic recombination techniques. When chemical synthesis is used, the protein is obtained through the polypeptide synthesis approach commonly used in the art. When genetic recombination techniques are applied, the nucleic acid code of the herbicide-resistant PPO protein will be inserted via an appropriate expression vector, and the above-mentioned vector will be transformed into a host cell. After culturing the host cell to express the target protein, the herbicide-resistant PPO protein then can be found and obtained in the host cell. After the protein being expressed in the selected host cell, commonly used biochemical methods are adopted for isolation. For example, isolation and purification is carried out by protein precipitants (salting-out), centrifugal separation, ultrasonic ablation, ultrafiltration, dialysis, molecular sieve chromatography (gel filtration), adsorption chromatography, ion exchange chromatography, affinity chromatography and other types of treatments. Usually, in order to obtain isolated proteins of high purity, several of the above-mentioned methods can be used in combination.

The herbicide-resistant PPO nucleic acid molecules can be isolated and prepared by standard molecular biology methods. For example, chemical synthesis or recombination techniques. It can be carried out by one of commercialized techniques.

The obtained PPO proteins described above can be transferred to plants for enhancing herbicide resistance of plants.

The above-mentioned herbicide-resistant PPO gene can be introduced into plants according to the methods commonly used in the art, by transgenic or gene editing operations with appropriate plant transformation expression vectors.

The selection of any appropriate promoter is a common practice in the art when performing genetic modification or gene editing in plants including vectors. For example, promoters commonly used in genetic modification or gene editing in plants include, but are not limited to, the SP6 promoter, T7 promoter, T3 promoter, PM promoter, maize ubiquitin promoter, cauliflower mosaic virus (CaMV) 35S promoter, nopaline synthase (nos) promoter, figwort mosaic virus 35S promoter, sugarcane bacilliform virus promoter, commelina yellow mottle virus promoter, light inducible promoter from the ribulose-1,5-ketose carboxylase (ssRUBISCO small subunit), rice cytoplast triose-phosphate isomerase (TPI) promoter, Arabidopsis adenine phosphoribosyl transferase (APRT) promoter, octopine synthase promoter and BCB (blue copper-binding protein) promoter.

Plant transgenic or gene editing vectors comprise a polyadenylation signal sequence causing the 3′-terminal polyadenylation. Examples include, but are not limited to, the NOS 3′-terminal derivatives of the nopaline synthase gene of Agrobacterium, octopine synthase 3′-terminal derivatives of the octopine synthase gene of Agrobacterium, 3′-terminal of the tomato or potato proteinase inhibitor I or II gene, CaMVPoly A signal sequence, 3′-terminal of the rice α-amylase gene and 3′-terminal of the phaseoline gene.

The above-mentioned transgenic vectors are to express the herbicide-resistant PPO gene in chloroplasts, and the transit peptides marked on the chloroplasts can be connected to the 5′-terminus of the PPO gene.

The vector also comprises the genetic code of a selectable marker that is a reporter molecule, and examples of selectable markers include, but are not limited to, antibiotics (e.g., neomycin, carbenicillin, kanamycin, spectinomycin, hygromycin, bleomycin, chloramphenicol, etc.) or herbicide-resistant (glyphosate, glufosinate-ammonium, glufosinate, etc.) genes.

Methods of vector transformation include introducing recombinant plasmids into plants using methods such as Agrobacterium-mediated transformation, electroporation, microparticle bombardment, polyethylene glycol (PEG)-medium absorption, etc.

The recipients of plant transformation in the present invention include plant cells (including suspension culture cells), protoplasts, calli, hypocotyls, seeds, cotyledons, shoots and mature plant bodies.

The scope of transgenic or gene-edited plants includes not only the plant body obtained at the time of gene introduction, but also clones and progeny thereof (T1 generation, T2 generation or subsequent generations). For example, transgenic or gene-edited plants comprising the coding nucleotide sequence of the PPO2 polypeptide provided in the present invention, which is tolerant to PPO inhibitor herbicides; progeny obtained through sexual and asexual propagation comprising the above-mentioned coding nucleotide sequence of the PPO2 polypeptide, which is tolerant to PPO inhibitor herbicides; and plants having inherited herbicide resistance traits are also included. The scope of the present invention also includes all mutants and variants, by crossing and fusion of the above-mentioned transgenic or gene-edited plants, exhibiting the characteristics of the first-generation transgenic or gene-edited plants. The scope of the present invention also includes a part of plants, such as seed, flower, stem, fruit, leaf, root, tuber, tuberous stem, which are derived from plants that have been modified by transgenosis or gene editing in advance by the methods mentioned in the present invention or from progeny thereof, and at least consist of a portion of transgenic or gene-edited cells.

The present invention also provides a method for controlling weeds in plant cultivation sites, wherein the plant includes the aforementioned plant or a plant prepared by the aforementioned method, and the method includes applying a herbicidally effective amount of protoporphyrinogen oxidase inhibitor herbicides to the cultivation site.

In one embodiment, a protoporphyrinogen oxidase inhibitor herbicide is used for controlling weeds.

In another embodiment, two or more protoporphyrinogen oxidase inhibitor herbicides are used sequentially or simultaneously for controlling weeds.

In yet another embodiment, the protoporphyrinogen oxidase inhibitor herbicide is applied in combination with one or more additional herbicides.

In the present invention, the term “site” includes a field where the plants of the present invention are cultivated such as soil, and also includes, for example, a plant seed, plant seedling as well as a grown plant. The term “herbicidally effective amount” refers to an amount of a herbicide sufficient to affect the growth or development of a target weed, for example, prevent or inhibit the growth or development of a target weed, or kill the weed. Advantageously, the herbicidally effective amount does not significantly affect the growth and/or development of the plant seeds, plant seedlings or plants of the present invention. Such herbicidally effective amounts can be determined by those skilled in the art through conventional experiments.

The present invention may be implemented in a variety of different forms and the implementation methods should not be limited to those set forth herein. The embodiments given herein are provided to achieve a thorough and complete disclosure, allowing hose skilled in the art to fully understand the scope of the invention. The same reference numbers refer to the same elements in the present invention.

As used herein, “first”, “second” and “third” are intended to describe a variety of different factors and components, which are not limited by the terms. These terms are used to distinguish a factor and component from another.

The terminology used herein is for the purpose of describing particular embodiments rather than setting limitations. Unless the context clearly dictates otherwise, “a”, “an”, and “the” used in the Chinese and English versions of the above content also include their plural forms. The terms “comprises” and/or “comprising”, or “includes” and/or “including” as used herein specifically refer to the presence of features, factors and/or components described herein, and do not exclude the presence and addition of one or more other features, factors and/or components. The term “and/or” used above includes all items in one or more combined lists.

The present invention has been illustrated in detail by a series of embodiments, however, the present invention is not limited to the disclosed embodiments. Any numerical changes, substitutions, replacements, and so on, which are within the scope of the present invention, are not stated herein, or may be modified as needed.

The beneficial effects of the present invention are as follows: the mutant forms may reduce the inhibiting effect of protoporphyrinogen oxidase inhibitor herbicides on protoporphyrinogen oxidase that comprises the mutant forms, but meanwhile, the mutations do not decrease the catalytic activity of protoporphyrinogen oxidase itself; modifying the endogenous protoporphyrinogen oxidase (PPO2) in plants into these mutant forms by means of gene editing or introducing the gene that comprises these protoporphyrinogen oxidase mutants into plants by transgenic means can significantly increase resistance of plants to protoporphyrinogen oxidase inhibitor herbicides; it may be used on plants, including economic crops, and applied according to herbicide resistance characteristics and herbicide selection, so as to achieve the purpose of controlling weed growth economically.

The present invention will be further illustrated below with reference to embodiments. All methods and procedures described in these embodiments are provided as examples and should not be construed as limited.

### Example 1

### Cloning of Rice Protoporphyrinogen Oxidase PPO2 Gene

The rice (Oryza sativa Japonica Group) mitochondrial protoporphyrinogen oxidase (PPO2) gene is located on chromosome 4, of which the NCBI gene symbol is LOC4336237. According to its cDNA sequence and pET15b (Novagen) vector sequence, the primers were designed and synthesized, and a DNA expression vector of the wild-type rice OsPPO2 coding region (SEQ ID NO: 1) was successfully constructed, which was named pET15b-OsPPO2 WT.

### Example 2

Testing Tolerance of Different Rice OsPPO2 Mutants to Compound a Using PPO-Defective Escherichia coli (ΔhemG)

Tolerance of rice OsPPO2 to herbicides was tested using PPO-defective Escherichia coli (ΔhemG). The ΔhemG is a strain of Escherichia coli (E. coli) defective in the hemG-type PPO gene and having kanamycin resistance (Watanabe N, Che F S, Iwano M, et al. Dual Targeting of Spinach Protoporphyrinogen Oxidase II to Mitochondria and Chloroplasts by Alternative Use of Two In-frame Initiation Codons [J]. Journal of Biological Chemistry, 2001, 276 (23): 20474-20481.). The cloned plasmid of rice OsPPO2 prepared above was added into ΔhemG competent cells and transformed by electroporation, so that the knockout strain regained PPO activity and could grow on ordinary LB agar media added with ampicillin and kanamycin.

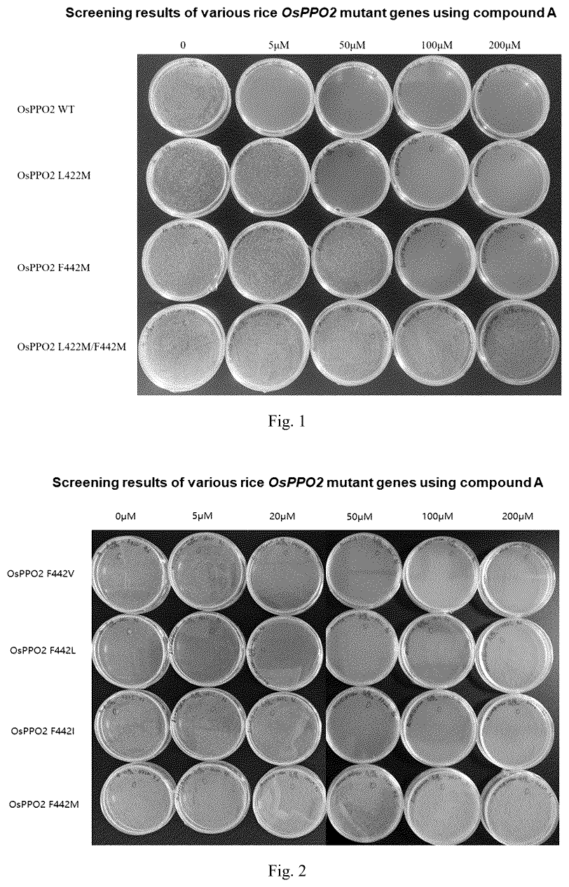

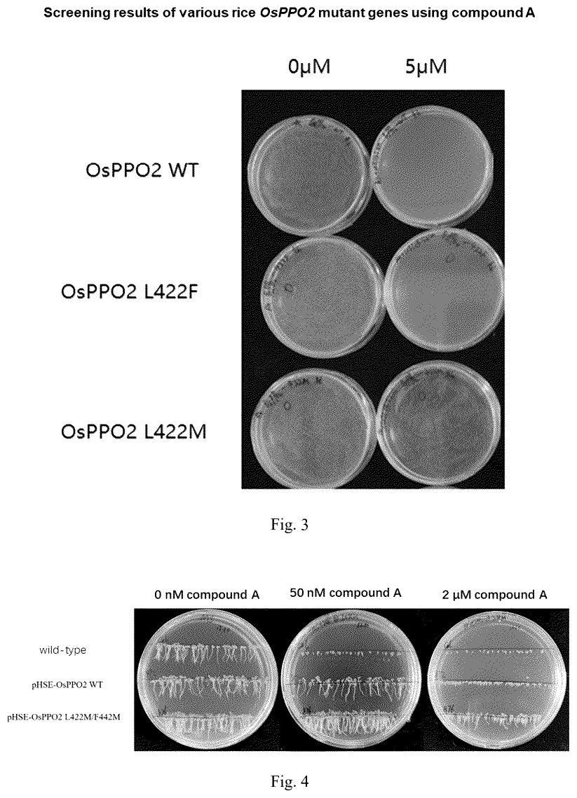

In order to test tolerance of different rice OsPPO2 mutants to PPO inhibitor herbicides, the tolerance tests of L422M (SEQ ID NO: 2), F442M (SEQ ID NO: 3) and L422M/F442M (SEQ ID NO: 4) to compound A were carried out by the E. coli screening system. The screening results are shown in FIGS. 1-3. Compared with the wild type and other published mutants, rice PPO2-L422M, F442M and L422M/F442M mutants all had certain tolerance to compound A, wherein the L422M/F442M mutation site combination was able to grow normally when treated with 200 μM compound A, exhibiting higher tolerance.

### Example 3

Tolerance of Rice OsPPO2 L422M/F442M Mutants Overexpressed in Arabidopsis thaliana to Different Compounds

In order to rapidly verify the tolerance of rice OsPPO2 L422M/F442M mutants to different compounds, vectors for overexpressing wild-type and mutant rice PPO2 genes were constructed by conventional methods and overexpressed in Arabidopsis thaliana.

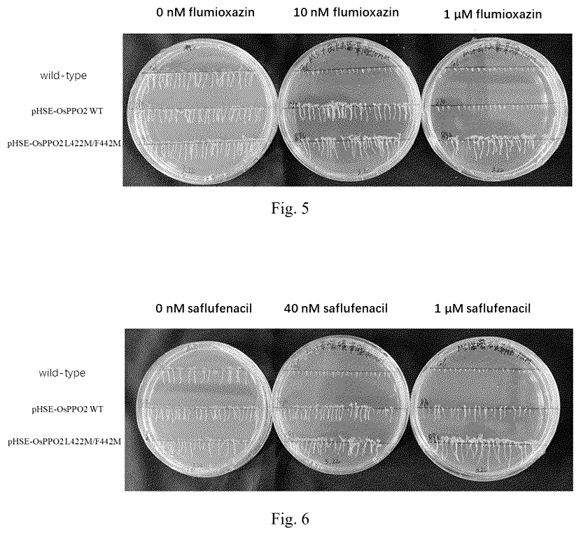

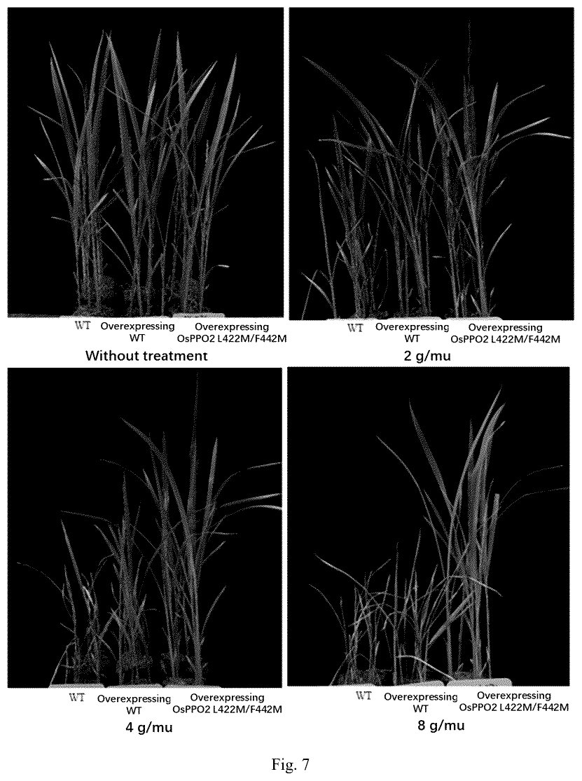

The obtained Arabidopsis thaliana seeds overexpressing rice OsPPO2 mutants and wild types were tested for resistance on MS media (petri dishes) that contained PPO inhibitor herbicidal compounds of various concentrations. As shown in FIGS. 4-6, compared with the wild-type Arabidopsis thaliana, the plants overexpressing rice OsPPO2 L422M/F442M and OsPPO2 WT both had certain tolerance/resistance to compound A, flumioxazin and saflufenacil. The resistance levels of the two were similar at the treatment concentrations (50 nM, 10 nM and 40 nM, respectively), however at higher application concentrations (2 μM, 1 μM and 1 μM, respectively), the plants overexpressing rice OsPPO2 L422M/F442M mutants still exhibited resistance, while the plants overexpressing OsPPO2 WT had no difference from the wild-type Arabidopsis thaliana controls, indicating that the tolerance level to PPO inhibitor herbicidal compounds was higher when the OsPPO2 L422M/F442M mutant was overexpressed in Arabidopsis thaliana.

### Example 4

### Overexpressing Rice OsPPO2 L422M/F442M Mutants to Acquire Herbicide Resistance

In order to further test the tolerance of the obtained mutants to herbicides in plant bodies, the rice OsPPO2 L422M/F442M mutants were overexpressed in rice.

The T0-generation rice seedlings overexpressing rice OsPPO2 L422M/F442M and OsPPO2 WT were sprayed with compound A of various concentrations for resistance test. As shown in FIG. 7, compared with the wild-type Jinjing 818, the plants overexpressing rice OsPPO2 L422M/F442M and OsPPO2 WT both had certain tolerance/resistance to compound A. The resistance levels of the two were similar at the application concentration of 2 g/mu (1 mu=1/15 hectare), however under the condition of a higher application concentration of 8 g/mu, the plant overexpressing OsPPO2 L422M/F442M still exhibited resistance, while the leaves of the plant overexpressing OsPPO2 WT appeared to die, which had no difference from that of the wild-type control, indicating that the tolerance level of rice to compound A was higher when OsPPO2 L422M/F442M was overexpressed.

### Example 5

### Gene-Editing Rice to Resist PPO Inhibiting Herbicides

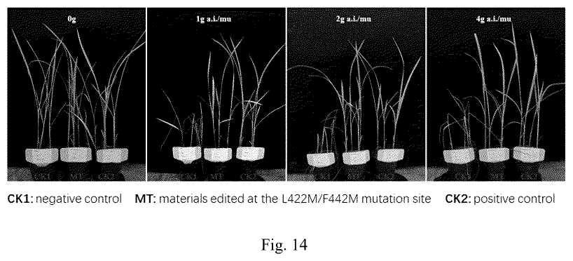

In order to acquire non-transgenic rice that is herbicide-resistant, CRISPR/cas9-mediated homologous replacement was performed on the above-mentioned L422M/F442M mutation site combination. It was identified that a homozygous OsPPO2 L422M/F442M rice strain with homologous replacement was successfully obtained, and the sequencing results are shown in FIG. 13. In order to further verify the tolerance of the above-mentioned gene-edited homozygous rice seedlings to compound A, the rice seedlings (4-leaf stage) were treated with 1 g, 2 g and 4 g/mu of compound A. Compared with the wild-type strain, the strain with homologous replacement at the L422M/F442M site could still survive at the dose of 4 g/mu, while the wild-type strain suffered severe phytotoxicity at the dose of 1 g/mu, and died eventually (see FIG. 14). Among them, the percentage statistics of resistance are shown in Table 1, and main weeds in rice fields such as Echinochloa crusgalli, Leptochloa chinensis, Monochoria vaginalis, Sagittaria trifolia, Alisma plantago-aquatica and the like all died at the dose of 1 g/mu. In addition, it was found after many tests that when other PPO inhibiting herbicides such as saflufenacil, flumioxazin, oxyfluorfen, pyraclonil, carfentrazone, Epyrifenacil, sulfentrazone, tiafenacil, fomesafen, trifludimoxazin,

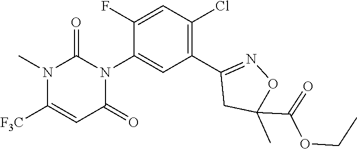 [SMILES: CCOC(=O)C1(C)CC(c2cc(-n3c(=O)cc(C(F)(F)F)n(C)c3=O)c(F)cc2Cl)=NO1]

were applied, it also had excellent crop safety and was able to create better selectivity on crops.

TABLE 1
Statistical results of percentage of resistance
of materials edited at L422M/F442M mutation site

| | Number of gene-edited rice | |
|---|---|---|
| Sample size | seedlings resistant to | Percentage of |
| (strain) | compound A (strain) | resistant seedlings |
| 163 | 112 | 68.7% |

### Example 6

### Verification of Tolerance to Compound a Caused by Mutations in Maize and Soybean PPO2 Genes Corresponding to the Rice OsPPO2 L422M/F442M Site

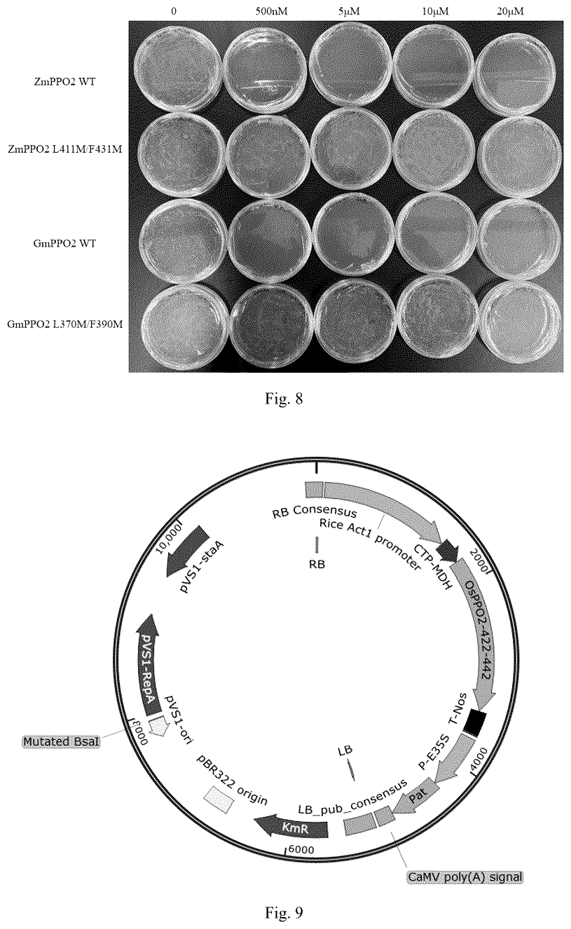

In order to verify whether the mutations corresponding to the rice PPO2 L422M/F442M site carried out in other plants can also produce herbicide resistance, the PPO2 gene of monocotyledonous maizes (ZmPPO2 L413M/F433M) and the PPO2 gene of dicotyledonous soybeans (GmPPO2 L370M/F390M) corresponding to the rice OsPPO2 L422M/F442M site were also tested in the E. coli screening system and screened with LB media containing herbicidal compound A to observe growth inhibition. As shown in FIG. 8, respectively compared with the wild-type ZmPPO2-WT (SEQ ID NO: 5) and GmPPO2-WT (SEQ ID NO: 7), the strains with mutated maize and soybean PPO2 genes had certain tolerance/resistance to compound A and grew normally on plates containing 500 nM compound A without inhibition. Moreover, as the concentration of compound A increased, the mutants all exhibited stronger tolerance, indicating that the mutations corresponding to the rice PPO2 L422M/F442M site in different plants had the same effect on herbicide tolerance.

### Example 7

### Overexpressing Rice OsPPO2 L422M/F442M Mutants in Maizes to Acquire Herbicide Resistance

In order to further test the tolerance of the obtained mutants to herbicides in other plant bodies, the rice OsPPO2 L422M/F442M mutants were overexpressed in maizes.

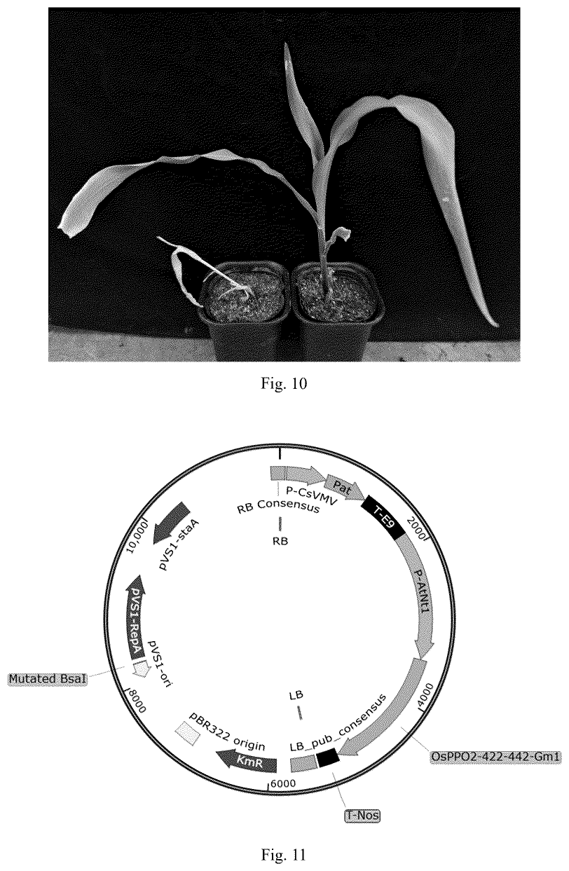

As shown in FIG. 9, the maize transgenic recombinant vector (SEQ ID NO: 22) was constructed and transformed into maizes by Agrobacterium-mediated transformation with immature embryos, and the transformants were screened using glufosinate-ammonium to obtain the transgenic maize seedlings that overexpressed rice PPO2 422M/442M in maizes. The TO-generation maize seedlings overexpressing rice OsPPO2 L422M/F442M were sprayed with compound A for resistance test.

As shown in FIG. 10, compared with the wild-type maize, overexpression of rice OsPPO2 L422M/F442M in the maize had certain tolerance/resistance to compound A. Under the condition of application concentration of 8 g/mu, the maize plant overexpressing OsPPO2 L422M/F442M still exhibited resistance, while the leaves of the wild-type plant appeared to die, indicating that the tolerance level of maizes to compound A increased when OsPPO2 L422M/F442M was overexpressed.

### Example 8

### Overexpressing Rice OsPPO2 L422M/F442M Mutants in Soybeans to Acquire Herbicide Resistance

As shown in FIG. 11, the soybean transgenic recombinant vector (SEQ ID NO: 23) was constructed and transformed into soybeans by Agrobacterium-mediated transformation with immature embryos, and the transformants were screened using glufosinate-ammonium to obtain the transgenic soybean seedlings overexpressing rice PPO2 422M/442M in soybeans. The TO-generation soybean seedlings overexpressing rice OsPPO2 L422M/F442M were sprayed with compound A for resistance test.

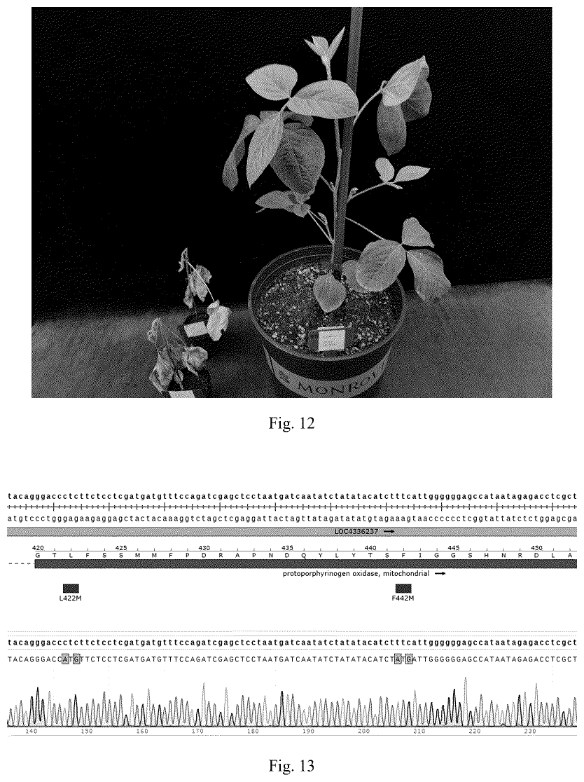

As shown in FIG. 12, compared with the wild-type soybean, overexpression of rice OsPPO2 L422M/F442M in the soybean had certain tolerance/resistance to compound A. Under the condition of application concentration of 12 g/mu, the soybean plant overexpressing OsPPO2 L422M/F442M still exhibited resistance, while the leaves of the wild-type plant appeared to die, indicating that the tolerance level of soybeans to compound A increased when OsPPO2 L422M/F442M was overexpressed.

Meanwhile, it was found after many tests that the introduction of the gene of the present invention into model plants, such as Arabidopsis thaliana, Brachypodium distachyon and the like, resulted in corresponding increases in drug resistance to PPO inhibiting herbicides. In addition, editing the above-mentioned mutation sites and combinations thereof by the CRISPR/Cpf1 system was also in the application. Thus, it may be known that transforming it into other aforementioned plants via transgenosis or gene editing would also produce corresponding resistance traits, which have good industrial value.

All publications and patent applications mentioned in the specification are hereby incorporated herein by reference, just as each publication or patent application is separately or particularly incorporated by reference into herein.

Although the foregoing invention has been described in detail through illustrations and examples for clear understanding, obviously some changes and modifications can be implemented within the scope of claims attached. Such changes and modifications are within the scope of the present invention.

1. A PPO2 polypeptide which is tolerant to PPO inhibitor herbicides or a bioactive fragment thereof, comprising an amino acid sequence having the following mutation(s) compared with the amino acid sequence as set forth in SEQ ID NO: 1: the amino acid corresponding to position 422 in the amino acid sequence set forth in SEQ ID NO: 1 is mutated from leucine into methionine, and/or the amino acid at position 442 is mutated from phenylalanine into methionine:
comprising an amino acid sequence having the following mutation(s) compared with the amino acid sequence as set forth in SEQ ID NO: 5: the amino acid corresponding to position 413 in the amino acid sequence set forth in SEQ ID NO: 5 is mutated from leucine into methionine, and/or the amino acid at position 433 is mutated from phenylalanine into methionine; or,
comprising an amino acid sequence having the following mutation(s) compared with the amino acid sequence as set forth in SEQ ID NO: 7: the amino acid corresponding to position 370 in the amino acid sequence set forth in SEQ ID NO: 7 is mutated from leucine into methionine, and/or the amino acid at position 390 is mutated from phenylalanine into methionine.

2. The PPO2 polypeptide or its bioactive fragment according to claim 1, wherein the amino acid sequence further has at least 80%, at least 85%, at least 90%, at least 95%, at least 96%, at least 97%, at least 98% or at least 99% sequence identity to the amino acid sequences set forth in SEQ ID NO: 1, 5 or 7, respectively.

3. The PPO2 polypeptide or its bioactive fragment according to claim 1, comprising an amino acid sequence that has at least 92%, at least 95%, at least 96%, at least 97%, at least 98%, at least 99% or 100% sequence identity to an amino acid sequence selected from the group consisting of SEQ ID NO: 2, 3, 4, 6 or 8.

4. An isolated polynucleotide comprising a nucleic acid sequence selected from:
(1) a nucleic acid sequence encoding the PPO2 polypeptide or its bioactive fragment according to claim 1, or a partial sequence or complementary sequence thereof;
(2) a nucleic acid sequence set forth in SEQ ID NO: 11 or 20, or a complementary sequence thereof;
(3) a nucleic acid sequence that hybridizes to the sequence set forth in (1) or (2) under stringent conditions; and/or
(4) a nucleic acid sequence encoding the same amino acid sequence as the sequence set forth in (1) or (2) due to degeneracy of the genetic code, or a complementary sequence thereof;
preferably, the polynucleotide is a DNA molecule.

5. A plant genome comprising the polynucleotide according to claim 4; optionally, wherein the plant is a monocotyledonous or dicotyledonous plant.

6. A vector construct comprising the polynucleotide according to claim 4 and a homologous or a non-homologous promoter operably linked thereto.

7. A vector construct comprising:
(1) a gene of which the nucleotide sequence is as set forth in SEQ ID NO: 11 and a gene of which the nucleotide sequence is as set forth in SEQ ID NO: 14;
(2) a gene of which the nucleotide sequence is as set forth in SEQ ID NO: 17 and a gene of which the nucleotide sequence is as set forth in SEQ ID NO: 20;
(3) two expression cassettes in tandem, wherein one expression cassette comprises a rice Act1 promoter of which the nucleotide sequence is as set forth in SEQ ID NO: 9, a CTP-MDH chloroplast-localized peptide of which the nucleotide sequence is as set forth in SEQ ID NO: 10, a gene of which the nucleotide sequence is as set forth in SEQ ID NO: 11, and a T-NOS terminator of which the nucleotide sequence is as set forth in SEQ ID NO: 12; the other expression cassette comprises a P-E35S promoter of which the nucleotide sequence is as set forth in SEQ ID NO: 13, a gene of which the nucleotide sequence is as set forth in SEQ ID NO: 14, and a CaMV poly (A) signal terminator of which the nucleotide sequence is as set forth in SEQ ID NO: 15; or,
(4) two expression cassettes in tandem, wherein one expression cassette comprises a P-CsVMV promoter of which the nucleotide sequence is as set forth in SEQ ID NO: 16, a gene of which the nucleotide sequence is as set forth in SEQ ID NO: 17, and a T-E9 terminator of which the nucleotide sequence is as set forth in SEQ ID NO: 18: the other expression cassette comprises a P-AtNt1 promoter of which the nucleotide sequence is as set forth in SEQ ID NO: 19, a gene of which the nucleotide sequence is as set forth in SEQ ID NO: 20, and a T-Nos terminator of which the nucleotide sequence is as set forth in SEQ ID NO: 21;
preferably, the nucleotide sequence of the vector construct is as set forth in SEQ ID NO: 22 or SEQ ID NO: 23.

8. A host cell comprising the polynucleotide according to claim 4 or a vector construct which comprises the polynucleotide and a homologous or non-homologous promoter operably linked thereto; preferably, the host cell is a plant cell; optionally, wherein the plant cell is a monocotyledonous or dicotyledonous plant cell.

9. A production method for plant cells capable of producing or increasing tolerance to protoporphyrinogen oxidase inhibitor herbicides, which includes using a gene editing approach to produce the polynucleotide according to claim 4 in plant cells or using a transgenic approach to introduce the polynucleotide or a vector construct which comprises the polynucleotide and a homologous or non-homologous promoter operably linked thereto, into plant cells; optionally, wherein the plant cell is a monocotyledonous or dicotyledonous plant cell.

10. A production method for plants capable of producing or increasing tolerance to protoporphyrinogen oxidase inhibitor herbicides and a plant produced by the method, wherein the method includes regenerating the plant cells according to claim 8 into plants; optionally, wherein the plant is a monocotyledonous or dicotyledonous plant.

11. A method for conferring or increasing tolerance to protoporphyrinogen oxidase inhibitor herbicides in plants, which includes introducing a modification into a gene that encodes a protein having protoporphyrinogen oxidase activity to produce the PPO2 polypeptide or its bioactive fragment according to claim 1; optionally, wherein the plant is a monocotyledonous or dicotyledonous plant.

12. A method of producing or increasing tolerance of plant cells, plant tissues, plant parts or plants to protoporphyrinogen oxidase herbicides, comprising expressing the PPO2 polypeptide or its bioactive fragment according to claim 1 in the plant cells, plant tissues, plant parts or plants;
or, comprising crossing a plant that expresses the PPO2 polypeptide or its bioactive fragment with another plant, as well as screening plants or parts thereof that are capable of producing or increasing tolerance to protoporphyrinogen oxidase herbicides;
or, comprising performing gene editing on a protein that has protoporphyrinogen oxidase activity of the plant cells, plant tissues, plant parts or plants, so as to express the PPO2 polypeptide or its bioactive fragment therein;
optionally, wherein the plant is a monocotyledonous or dicotyledonous plant.

13. (canceled)

14. A method for controlling weeds in plant cultivation sites, wherein the plant includes a plant prepared by the method according to claim 10, and the method includes applying a herbicidally effective amount of protoporphyrinogen oxidase inhibitor herbicides to the cultivation site; optionally, wherein the protoporphyrinogen oxidase inhibitor herbicide is applied in combination with one or more additional herbicides.

15-16. (canceled)

17. The method according to claim 12, wherein the protoporphyrinogen oxidase inhibitor herbicides are selected from one or two or more of the following types of compounds: pyrimidinediones, diphenyl-ethers, phenylpyrazoles, N-phenylphthalimides, thiadiazoles, oxadiazoles, triazolinones, oxazolidinediones and others; preferably,
(1) pyrimidinedione herbicides include butafenacil, saflufenacil, benzfendizone, tiafenacil, [3-[2-chloro-4-fluoro-5-(1-methyl-6-trifluoromethyl-2,4-dioxo-1,2,3,4-tetrahydropyrimidin-3-yl) phenoxy]-2-pyridyloxy] ethyl acetate, 1-methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione, 3-[7-chloro-5-fluoro-2-(trifluoromethyl)-1H-benzimidazol-4-yl]-1-methyl-6-(trifluoromethyl)-1H-pyrimidine-2,4-dione, flupropacil, 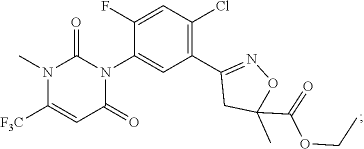 [SMILES: CCOC(=O)C1(C)CC(c2cc(-n3c(=O)cc(C(F)(F)F)n(C)c3=O)c(F)cc2Cl)=NO1]
(2) diphenyl-ether herbicides include fomesafen, oxyfluorfen, aclonifen, lactofen, chlomethoxyfen, chlornitrofen, fluoroglycofen-ethyl, acifluorfen or acifluorfen-sodium, bifenox, ethoxyfen, ethoxyfen-ethyl, fluoronitrofen, furyloxyfen, nitrofluorfen and halosafen;
(3) phenylpyrazole herbicides include pyraflufen-ethyl and fluazolate;
(4) N-phenylphthalimide herbicides include flumioxazin, cinidon-ethyl, flumipropyn and flumiclorac-pentyl;
(5) thiadiazole herbicides include fluthiacet-methyl, fluthiacet and thidiazimin;
(6) oxadiazole herbicides include oxadiargyl and oxadiazon;
(7) triazolinone herbicides include carfentrazone, carfentrazone-ethyl, sulfentrazone, azafenidin and bencarbazone;
(8) oxazolidinedione herbicides include pentoxazone;
(9) other herbicides include pyraclonil, flufenpyr-ethyl, profluazol, trifludimoxazin, N-ethyl-3-(2,6-dichloro-4-trifluoromethylphenoxy)-5-methyl-1H-pyrazole-1-carboxamide, N-tetrahydrofurfuryl-3-(2,6-dichloro-4-trifluoromethylphenoxy)-5-methyl-1H-pyrazole-1-carboxamide, N-ethyl-3-(2-chloro-6-fluoro-4-trifluoromethylphenoxy)-5-methyl-1H-pyrazole-1-carboxamide, N-tetrahydrofurfuryl-3-(2-chloro-6-fluoro-4-trifluoromethylphenoxy)-5-methyl-1H-pyrazole-1-carboxamide, 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione, 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione, Methyl (E)-4-[2-chloro-5-[4-chloro-5-(difluoromethoxy)-1H-methyl-pyrazol-3-yl]-4-fluoro-phenoxy]-3-methoxy-but-2-enoate, phenylpyridines, benzoxazinone derivatives and the compounds represented by Formula I 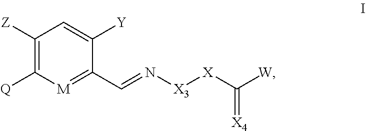
wherein,
Q represents 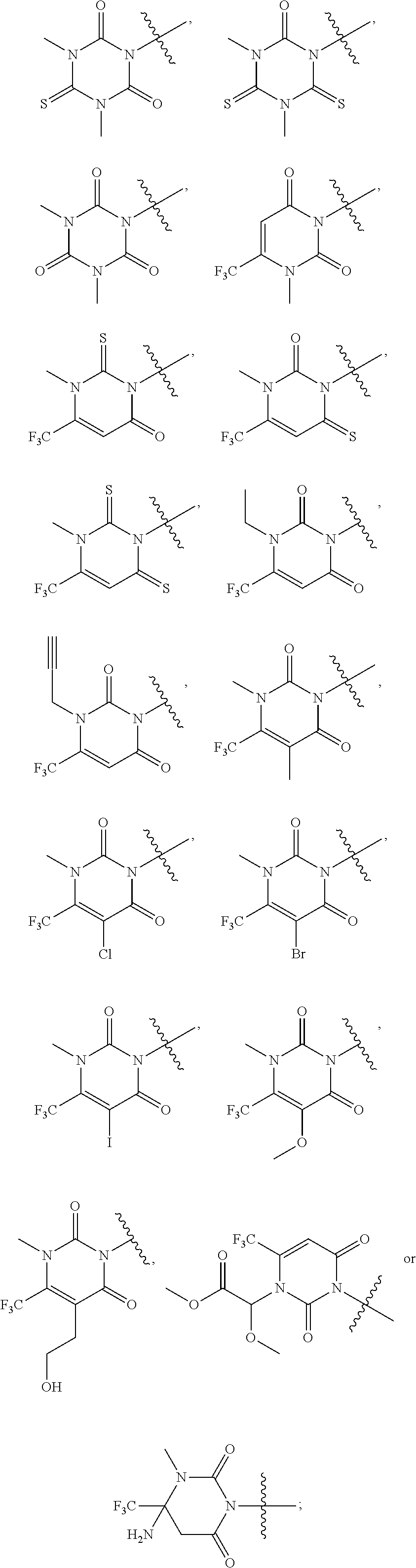
Y represents halogen, halo C1-C6 alkyl or cyano;
Z represents halogen;
M represents CH or N;
X represents —CX₁X₂—(C1-C6 alkyl)ₙ-, -(C1-C6 alkyl)-CX₁X₂—(C1-C6 alkyl)ₙ- or —(CH₂)ᵣ-, n represents 0 or 1, r represents an integer of 2 and above;
X₁ and X₂ independently represent hydrogen, halogen, C1-C6 alkyl, C2-C6 alkenyl, C2-C6 alkynyl, halo C1-C6 alkyl, halo C2-C6 alkenyl, halo C2-C6 alkynyl, C3-C6 cycloalkyl, C3-C6 cycloalkyl C1-C6 alkyl, C1-C6 alkoxy, C1-C6 alkylthio, hydroxyl C1-C6 alkyl, C1-C6 alkoxy C1-C6 alkyl, phenyl or benzyl, respectively;
X₃ and X₄ independently represent O or S, respectively;
W represents hydroxyl, C1-C6 alkoxy, C2-C6 alkenoxy, C2-C6 alkynoxy, halo C1-C6 alkoxy, halo C2-C6 alkenoxy, halo C2-C6 alkynoxy, C3-C6 cycloalkoxy, phenoxy, mercapto, C1-C6 alkylthio, C2-C6 alkenylthio, C2-C6 alkynylthio, halo C1-C6 alkylthio, halo C2-C6 alkenylthio, halo C2-C6 alkynylthio, C3-C6 cycloalkylthio, phenylthio, amino or C1-C6 alkylamino;
more preferably, Q represents 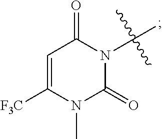
Y represents chlorine; Z represents fluorine; M represents CH; X represents —C*X₁X₂—(C1-C6 alkyl)n-, n represents 0; X₁ represents hydrogen; X₂ represents methyl; X₃ and X₄ independently represent O; W represents methoxy; wherein C* is the chiral center, and the compound is in R configuration.

18. A host cell comprising the vector construct according to claim 7; optionally, the host cell is a plant cell; optionally, wherein the plant cell is a monocotyledonous or dicotyledonous plant cell.

19. A production method for plant cells capable of producing or increasing tolerance to protoporphyrinogen oxidase inhibitor herbicides, which includes using a transgenic approach to introduce the vector construct according to claim 7 into plant cells; optionally, wherein the plant cell is a monocotyledonous or dicotyledonous plant cell.

20. A production method for plants capable of producing or increasing tolerance to protoporphyrinogen oxidase inhibitor herbicides and a plant produced by the method, wherein the method includes regenerating the plant cells according to claim 18 into plants; optionally, wherein the plant is a monocotyledonous or dicotyledonous plant.

21. A method for controlling weeds in plant cultivation sites, wherein the plant includes a plant prepared by the method according to claim 12, and the method includes applying a herbicidally effective amount of protoporphyrinogen oxidase inhibitor herbicides to the cultivation site; optionally, wherein the protoporphyrinogen oxidase inhibitor herbicide is applied in combination with one or more additional herbicides.

22. The method according to claim 14, wherein the protoporphyrinogen oxidase inhibitor herbicides are selected from one or two or more of the following types of compounds: pyrimidinediones, diphenyl-ethers, phenylpyrazoles, N-phenylphthalimides, thiadiazoles, oxadiazoles, triazolinones, oxazolidinediones and others; preferably,
(1) pyrimidinedione herbicides include butafenacil, saflufenacil, benzfendizone, tiafenacil, [3-[2-chloro-4-fluoro-5-(1-methyl-6-trifluoromethyl-2,4-dioxo-1,2,3,4-tetrahydropyrimidin-3-yl) phenoxy]-2-pyridyloxy] ethyl acetate, 1-methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione, 3-[7-chloro-5-fluoro-2-(trifluoromethyl)-1H-benzimidazol-4-yl]-1-methyl-6-(trifluoromethyl)-1H-pyrimidine-2,4-dione, flupropacil, 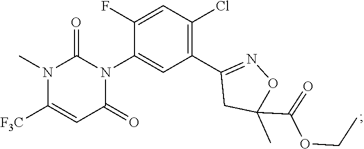 [SMILES: CCOC(=O)C1(C)CC(c2cc(-n3c(=O)cc(C(F)(F)F)n(C)c3=O)c(F)cc2Cl)=NO1]
(2) diphenyl-ether herbicides include fomesafen, oxyfluorfen, aclonifen, lactofen, chlomethoxyfen, chlornitrofen, fluoroglycofen-ethyl, acifluorfen or acifluorfen-sodium, bifenox, ethoxyfen, ethoxyfen-ethyl, fluoronitrofen, furyloxyfen, nitrofluorfen and halosafen;
(3) phenylpyrazole herbicides include pyraflufen-ethyl and fluazolate;
(4) N-phenylphthalimide herbicides include flumioxazin, cinidon-ethyl, flumipropyn and flumiclorac-pentyl;
(5) thiadiazole herbicides include fluthiacet-methyl, fluthiacet and thidiazimin;
(6) oxadiazole herbicides include oxadiargyl and oxadiazon;
(7) triazolinone herbicides include carfentrazone, carfentrazone-ethyl, sulfentrazone, azafenidin and bencarbazone;
(8) oxazolidinedione herbicides include pentoxazone;
(9) other herbicides include pyraclonil, flufenpyr-ethyl, profluazol, trifludimoxazin, N-ethyl-3-(2,6-dichloro-4-trifluoromethylphenoxy)-5-methyl-1H-pyrazole-1-carboxamide, N-tetrahydrofurfuryl-3-(2,6-dichloro-4-trifluoromethylphenoxy)-5-methyl-1H-pyrazole-1-carboxamide, N-ethyl-3-(2-chloro-6-fluoro-4-trifluoromethylphenoxy)-5-methyl-1H-pyrazole-1-carboxamide, N-tetrahydrofurfuryl-3-(2-chloro-6-fluoro-4-trifluoromethylphenoxy)-5-methyl-1H-pyrazole-1-carboxamide, 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione, 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione, Methyl (E)-4-[2-chloro-5-[4-chloro-5-(difluoromethoxy)-1H-methyl-pyrazol-3-yl]-4-fluoro-phenoxy]-3-methoxy-but-2-enoate, phenylpyridines, benzoxazinone derivatives and the compounds represented by Formula I 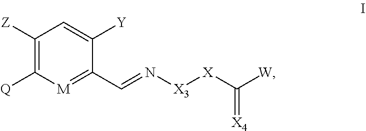
wherein,
Q represents 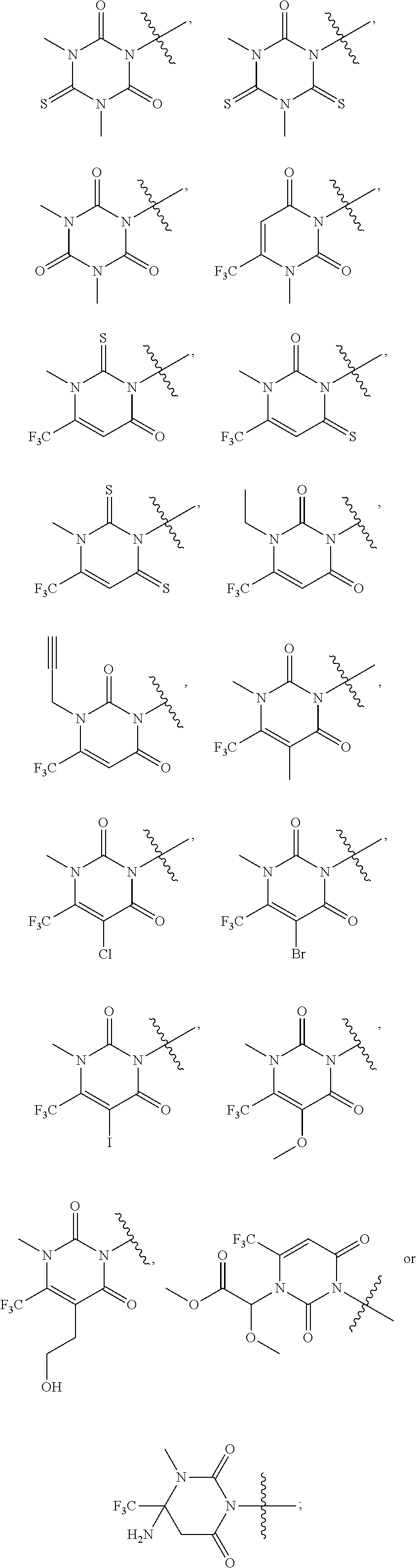
Y represents halogen, halo C1-C6 alkyl or cyano;
Z represents halogen;
M represents CH or N;
X represents —CX₁X₂—(C1-C6 alkyl)ₙ-, -(C1-C6 alkyl)-CX₁X₂—(C1-C6 alkyl)ₙ- or —(CH₂)ᵣ-, n represents 0 or 1, r represents an integer of 2 and above;
X₁ and X₂ independently represent hydrogen, halogen, C1-C6 alkyl, C2-C6 alkenyl, C2-C6 alkynyl, halo C1-C6 alkyl, halo C2-C6 alkenyl, halo C2-C6 alkynyl, C3-C6 cycloalkyl, C3-C6 cycloalkyl C1-C6 alkyl, C1-C6 alkoxy, C1-C6 alkylthio, hydroxyl C1-C6 alkyl, C1-C6 alkoxy C1-C6 alkyl, phenyl or benzyl, respectively;
X₃ and X₄ independently represent O or S, respectively;
W represents hydroxyl, C1-C6 alkoxy, C2-C6 alkenoxy, C2-C6 alkynoxy, halo C1-C6 alkoxy, halo C2-C6 alkenoxy, halo C2-C6 alkynoxy, C3-C6 cycloalkoxy, phenoxy, mercapto, C1-C6 alkylthio, C2-C6 alkenylthio, C2-C6 alkynylthio, halo C1-C6 alkylthio, halo C2-C6 alkenylthio, halo C2-C6 alkynylthio, C3-C6 cycloalkylthio, phenylthio, amino or C1-C6 alkylamino;
more preferably, Q represents 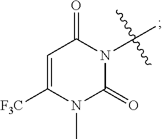
Y represents chlorine; Z represents fluorine; M represents CH; X represents —C*X₁X₂—(C1-C6 alkyl) n-, n represents 0; X₁ represents hydrogen; X₂ represents methyl; X₃ and X₄ independently represent O; W represents methoxy; wherein C* is the chiral center, and the compound is in R configuration.

23. The method according to claim 21, wherein the protoporphyrinogen oxidase inhibitor herbicides are selected from one or two or more of the following types of compounds: pyrimidinediones, diphenyl-ethers, phenylpyrazoles, N-phenylphthalimides, thiadiazoles, oxadiazoles, triazolinones, oxazolidinediones and others; preferably,
(1) pyrimidinedione herbicides include butafenacil, saflufenacil, benzfendizone, tiafenacil, [3-[2-chloro-4-fluoro-5-(1-methyl-6-trifluoromethyl-2,4-dioxo-1,2,3,4-tetrahydropyrimidin-3-yl) phenoxy]-2-pyridyloxy] ethyl acetate, 1-methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione, 3-[7-chloro-5-fluoro-2-(trifluoromethyl)-1H-benzimidazol-4-yl]-1-methyl-6-(trifluoromethyl)-1H-pyrimidine-2,4-dione, flupropacil, 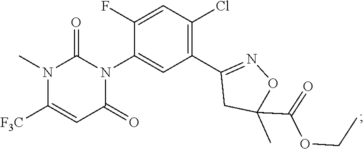 [SMILES: CCOC(=O)C1(C)CC(c2cc(-n3c(=O)cc(C(F)(F)F)n(C)c3=O)c(F)cc2Cl)=NO1]
(2) diphenyl-ether herbicides include fomesafen, oxyfluorfen, aclonifen, lactofen, chlomethoxyfen, chlornitrofen, fluoroglycofen-ethyl, acifluorfen or acifluorfen-sodium, bifenox, ethoxyfen, ethoxyfen-ethyl, fluoronitrofen, furyloxyfen, nitrofluorfen and halosafen;
(3) phenylpyrazole herbicides include pyraflufen-ethyl and fluazolate;
(4) N-phenylphthalimide herbicides include flumioxazin, cinidon-ethyl, flumipropyn and flumiclorac-pentyl;
(5) thiadiazole herbicides include fluthiacet-methyl, fluthiacet and thidiazimin;
(6) oxadiazole herbicides include oxadiargyl and oxadiazon;
(7) triazolinone herbicides include carfentrazone, carfentrazone-ethyl, sulfentrazone, azafenidin and bencarbazone;
(8) oxazolidinedione herbicides include pentoxazone;
(9) other herbicides include pyraclonil, flufenpyr-ethyl, profluazol, trifludimoxazin, N-ethyl-3-(2,6-dichloro-4-trifluoromethylphenoxy)-5-methyl-1H-pyrazole-1-carboxamide, N-tetrahydrofurfuryl-3-(2,6-dichloro-4-trifluoromethylphenoxy)-5-methyl-1H-pyrazole-1-carboxamide, N-ethyl-3-(2-chloro-6-fluoro-4-trifluoromethylphenoxy)-5-methyl-1H-pyrazole-1-carboxamide, N-tetrahydrofurfuryl-3-(2-chloro-6-fluoro-4-trifluoromethylphenoxy)-5-methyl-1H-pyrazole-1-carboxamide, 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione, 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione, Methyl (E)-4-[2-chloro-5-[4-chloro-5-(difluoromethoxy)-1H-methyl-pyrazol-3-yl]-4-fluoro-phenoxy]-3-methoxy-but-2-enoate, phenylpyridines, benzoxazinone derivatives and the compounds represented by Formula I 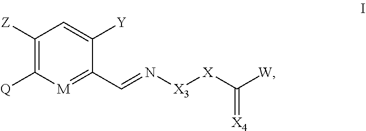
wherein,
Q represents 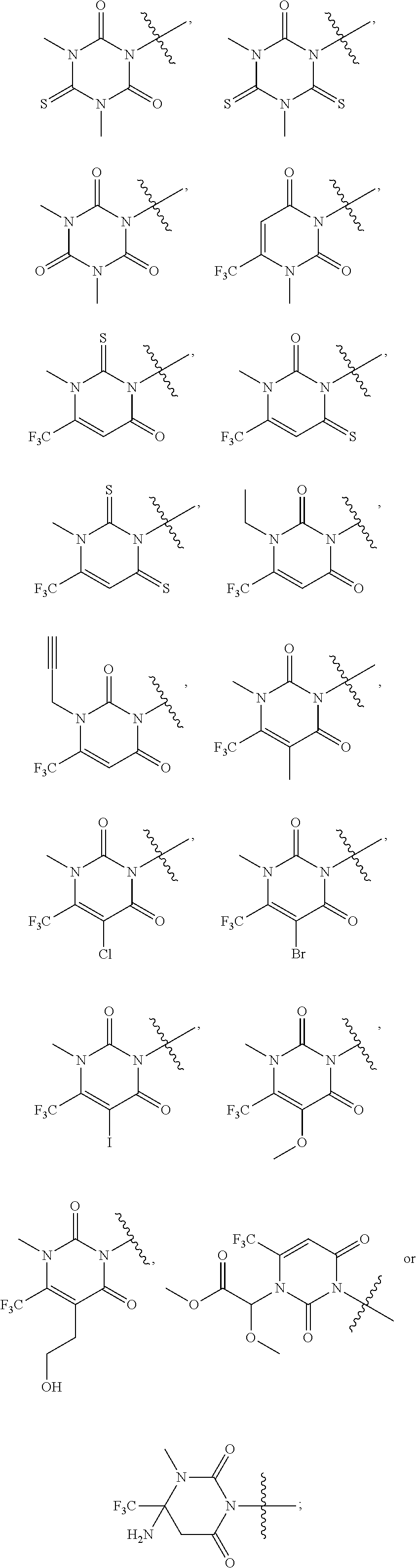
Y represents halogen, halo C1-C6 alkyl or cyano;
Z represents halogen;
M represents CH or N;
X represents —CX₁X₂—(C1-C6 alkyl)ₙ-, -(C1-C6 alkyl)-CX₁X₂—(C1-C6 alkyl)ₙ- or —(CH₂)ᵣ-, n represents 0 or 1, r represents an integer of 2 and above;
X₁ and X₂ independently represent hydrogen, halogen, C1-C6 alkyl, C2-C6 alkenyl, C2-C6 alkynyl, halo C1-C6 alkyl, halo C2-C6 alkenyl, halo C2-C6 alkynyl, C3-C6 cycloalkyl, C3-C6 cycloalkyl C1-C6 alkyl, C1-C6 alkoxy, C1-C6 alkylthio, hydroxyl C1-C6 alkyl, C1-C6 alkoxy C1-C6 alkyl, phenyl or benzyl, respectively;
X₃ and X₄ independently represent O or S, respectively;
W represents hydroxyl, C1-C6 alkoxy, C2-C6 alkenoxy, C2-C6 alkynoxy, halo C1-C6 alkoxy, halo C2-C6 alkenoxy, halo C2-C6 alkynoxy, C3-C6 cycloalkoxy, phenoxy, mercapto, C1-C6 alkylthio, C2-C6 alkenylthio, C2-C6 alkynylthio, halo C1-C6 alkylthio, halo C2-C6 alkenylthio, halo C2-C6 alkynylthio, C3-C6 cycloalkylthio, phenylthio, amino or C1-C6 alkylamino;
more preferably, Q represents 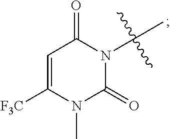 [SMILES: *n1c(=O)cc(C(F)(F)F)n(C)c1=O]
Y represents chlorine; Z represents fluorine; M represents CH; X represents —C*X₁X₂—(C1-C6 alkyl) n-, n represents 0; X₁ represents hydrogen; X₂ represents methyl; X₃ and X₄ independently represent O; W represents methoxy; wherein C* is the chiral center, and the compound is in R configuration.
